(19) United States
(12) Patent Application Publication
(10) Pub. No.: US 2026/0159485 A1
(43) Pub. Date: Jun. 11, 2026

(54) ANDROGEN RECEPTOR DEGRADERS FOR THE TREATMENT OF CASTRATION-RESISTANT PROSTATE CANCER AND USE THEREOF

(71) Applicants: AUTOTAC INC., Seoul (KR); SEOUL NATIONAL UNIVERSITY R&DB FOUNDATION, Seoul (KR)

(72) Inventors: Yong Tae KWON, Seoul (KR); Ki Woon SUNG, Seoul (KR); Tae Hyun BAE, Seoul (KR); Hyun Tae KIM, Seoul (KR); Jeong Eun NA, Seoul (KR); Kun Young KIM, Seoul (KR); Ha Kyoung KWON, Seoul (KR)

(73) Assignees: AUTOTAC INC., Seoul (KR); SEOUL NATIONAL UNIVERSITY R&DB FOUNDATION, Seoul (KR)

(21) Appl. No.: 18/706,634

(22) PCT Filed: Nov. 2, 2022
(86) PCT No.: PCT/KR2022/017075

§ 371 Date: May 1, 2024

Related U.S. Application Data
(60) Provisional application No. 63/274,577, filed on Nov. 2, 2021.

(51) Int. Cl. C07D 233/86; A61K 31/4166; A61K 31/496; A61P 35/00; C07D 417/14

(52) U.S. Cl. CPC C07D 233/86; A61K 31/4166; A61K 31/496; A61P 35/00; C07D 417/14

(57) ABSTRACT

Described herein novel compounds as androgen-receptor inhibitors for the treatment of prostate cancer and use thereof.

### TECHNICAL FIELD

The present invention relates to novel chemical compounds and pharmaceutically acceptable compositions thereof which show androgen receptor antagonizing and degrading activity.

### BACKGROUND ART

Prostate cancer is the most common incidence of cancer and the second leading cause of cancer death in Western men. When the cancer is confined locally, the disease can be cured by surgery or radiation. However, 30% of such cancer relapses with distant metastatic disease and others have advanced disease at diagnoses. Advanced disease is treated by castration and/or administration of anti-androgens, the so-called androgen deprivation therapy. Castration lowers the circulating levels of androgens and reduces the activity of androgen receptor (AR). Administration of anti-androgens blocks AR function by competing away androgen binding and therefore reduces the AR activity. Although initially effective, these treatments quickly fail and the cancer becomes hormone refractory or castration resistant.

Recently, overexpression of AR has been identified and validated as a cause of castration resistant prostate cancer. Overexpression of AR is sufficient to cause progression from hormone sensitive to this cancer, suggesting that better AR inhibitors than the current drugs can slow the progression of prostate cancer. It was demonstrated that AR and its ligand binding are necessary for growth of castration resistant prostate cancer, indicating that AR is still a target for this disease. It was also demonstrated that mutations of ligand binding domain accelerate anti-androgens from antagonists to agonists in castration resistant prostate cancer (an AR antagonist inhibits AR activity and an AR agonist stimulates AR activity). Data from this work explain why castration and anti-androgens fail to prevent prostate cancer progression and reveals un-recognized properties of castration resistant prostate cancer.

It is necessary to develop a next-generation drug form to which a new paradigm or mode of action is applied, especially for patients who have castration-resistant prostate cancer. The disclosed methods are directed to these and other important needs.

### DISCLOSURE OF INVENTION

### Technical Problem

This disclosure is for providing a next-generation drug form to which a new paradigm or mode of action is applied, especially for patients who have castration-resistant prostate cancer. The disclosed methods are directed to these and other important needs.

### Solution to Problem

The present invention relates to novel compounds of general formula (I) shown below, pharmaceutically acceptable salt, diasteromers, enantiomers, racemates, tautomers, prodrugs, hydrates, or solvates thereof:

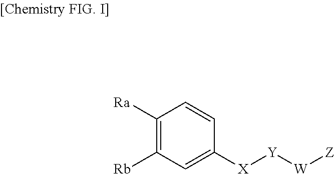

in formula (I),
wherein Ra and Rb are independently H or —O—CH₂—R1,
R1 is C6-C9 aryl optionally substituted with one or more halos,
X is —O—CH₂—CH(OH)—CH₂NH— or —CH₂—NH—;
Y is —(CH₂CH₂—O)ₙ₁—, —(CH₂)ₙ₂—O—, n1 and n2 are independently an integer of 1 to 6;
W is —(CH₂)ₙ₃—NH—C(═O)—, —(CH₂)ₙ₁—NH—C(═O)—(CH)ₙ₅—O—, —CH₂—CH₂—O—, —CH₂—C(═O)—, n3, n4 and n5 are independently an integer of 1 to 7, preferably, —CH₂CH₂—NH—C(═O)—, Z is

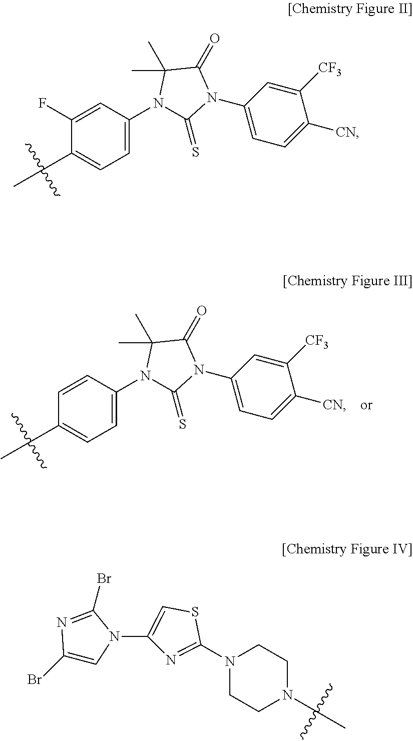 [SMILES: *c1ccc(N2C(=S)N(c3ccc(C#N)c(C(F)(F)F)c3)C(=O)C2(C)C)cc1F ; *c1ccc(N2C(=S)N(c3ccc(C#N)c(C(F)(F)F)c3)C(=O)C2(C)C)cc1 ; *N1CCN(c2nc(-n3cc(Br)nc3Br)cs2)CC1]

### Advantageous Effects of Invention

The disclosure can provide novel compounds as androgen-receptor inhibitors for the treatment of prostate cancer and use thereof.

### BRIEF DESCRIPTION OF DRAWINGS

FIG. 1a to FIG. 1e are results and graphs showing increase of the autophagy flux in androgen-responsive and unresponsive cell by AR inhibitor of the present disclosure.

FIG. 2a to FIG. 2c are results and graphs showing anticancer efficacy via degradation of androgen receptor: Xtandi vs. AUTOTAC (present invention). All Xtandi-AUTOTAC compounds (Compounds, A, B, C, D, E, F, G, H and I) efficiently degrade androgen receptor through autophagy

FIG. 3 is results and graph showing anticancer efficacy via inhibition of transcriptional activity of androgen receptor by forming aggregation with p62 in the cytosol.

FIG. 4 is graphs showing down-regulation of AR signaling pathway.

FIG. 5 is a graph showing that treatment of Xtandi-AUTOTAC induces prostate cancer cell death.

FIG. 6 is results and graph showing that Compound B prevents colony formation of prostate cancer cell.

FIG. 7 is results and graph showing that Compound B inhibits cell proliferation by blocking the cell cycle in G1 phase.

FIG. 8a to FIG. 8d are data showing Compound B efficiently inhibits tumor growth in Xenograft mouse model, and FIG. 8e is results showing Compound B degrades androgen receptor in tumor of mice.

FIG. 9a to FIG. 9d are data showing AR splice variant 7 (AR-V7) mediates Xtandi(Enzalutamide) resistance. Compound B degrades AR-V7 as well as wild type AR in enzalutamide resistant cell. Compound B can degrade AR-V7 by sequestering with wild type AR.

FIG. 10 is data showing Compound B inhibits tumor growth in AR-V7-expressing 22Rv1 xenograft model.

### BEST MODE FOR CARRYING OUT THE INVENTION

Hereinafter, the present invention will be described in more detail.

Unless otherwise defined, all technical terms used herein have the same meaning as commonly understood by one of ordinary skill in the art to which this invention belongs. Also, although the invention has been described in conjunction with specific methods and samples, their analogs or equivalents should be within the scope of the present invention. Furthermore, the numerical values set forth herein are considered to include the meaning of “about” unless explicitly stated. All publications and other references mentioned herein art hereby incorporated by reference in their entirety.

Prostate cancer is the second most common cause of cancer death in men in the US, and approximately one in every six American men will be diagnosed with the disease during his lifetime. In the early stages of prostate cancer, the cancer is localized to the prostate. In these early stages, treatment typically involves either surgical removal of the prostate or radiation therapy to the prostate or observation only with no active intervention therapy in some patients. In the early stages where the prostate cancer is localized and requires intervention, surgery or radiation therapy are curative by eradicating the cancerous cells. About 30% of the time these procedures fail, and the prostate cancer continues to progress, as typically evidenced by a rising PSA level. Men whose prostate cancer has progressed following these early treatment strategies are said to have advanced or recurrent prostate cancer. Treatment aimed at eradicating the tumor is unsuccessful in 30% of men, who develop recurrent disease that is usually manifest first as a rise in plasma prostate-specific antigen (PSA) followed by spread to distant sites.

Androgen receptor (AR) is a member of the steroid and nuclear receptor superfamily. Among this large family of proteins, only five vertebrate steroid receptors are known and include the androgen receptor, estrogen receptor, progesterone receptor, gluco-corticoid receptor, and mineralocorticoid receptor. AR is a soluble protein that functions as an intracellular transcriptional factor. AR function is regulated by the binding of androgens, which initiates sequential conformational changes of the receptor that affect receptor-protein interactions and receptor-DNA interactions.

Given that prostate cancer cells depend on androgen receptor (AR) for their proliferation and survival, these men are treated with agents that block production of testosterone (e.g., GnRH agonists), alone or in combination with anti-androgens (e.g., bicalutamide), which antagonize the effect of any residual testosterone on AR. The approach is effective as evidenced by a drop in PSA and regression of visible tumor (if present) in some patients; however, this is followed by regrowth as a castration resistant prostate cancer (CRPC) to which most patients eventually succumb. Recent studies on the molecular basis of CRPC have demonstrated that CRPC continues to depend on AR signaling and that a key mechanism of acquired resistance is an elevated level of AR protein (Nat. Med, 2004, 10, 33-39). AR targeting agents with activity in castration sensitive and castration resistant prostate cancer have great promise in treating this lethal disease.

Castration resistant prostate cancer (CRPC) is a lethal phenotype and almost all of patients will die from prostate cancer. Interestingly, while a small minority of CRPC does bypass the requirement for AR signaling, the vast majority of CRPC, though frequently termed “androgen independent prostate canceror” or “hormone refractory prostate cancer,” retains its lineage dependence on AR signaling.

As used herein, the term “Androgen receptor degrader (AR degrader)” are used inter-changeably herein and refer to an agent that interacts with the androgen receptor (A) such that it causes the AR to be sequestrated, degraded and thus down regulated.

The present invention provides novel compounds and pharmaceutically acceptable salts thereof that are useful for degrading AR, for inhibiting AR activity, and for treating diseases and disorders that are mediated by AR signaling, for example, prostate cancer, specifically castration resistant prostate cancer.

Conventional inhibitors such as enzalutamide, bicalutamide and abiraterone, acting through the ligand binding domain (LBD), fail to inhibit growth driven by the N-terminal domain (NTD)-dependent constitutively active AR splice variants (AR-Vs) such as AR-V7, the most prominent AR·Vs. These conventional inhibitors cannot inhibit CRPC growth which is dependent on constitutively active AR including AR-SV lacking the LBD such as AR-V7. However, the AR degrader compound of the present invention can treat untreatable with any other inhibitors and antagonists by its unique properties of degrading AR and AR-Vs through autophagy, inhibits transcriptional activity of AR by sequestering, down regulating expression of AR target genes.

The present invention also provides pharmaceutical compositions comprising at least one of the compounds of Formula (I) together with a pharmaceutically acceptable carrier, diluent or excipient therefor.

The present invention provides compositions and methods for modulating the activity of the AR. In one aspect, the present invention provides compounds which act as inhibitors of ARs.

In one embodiment, provided herein is a compound of Formula (I) shown below, pharmaceutically acceptable salt, diasteromers, enantiomers, racemates, tautomers, prodrugs, hydrates, or solvates thereof:

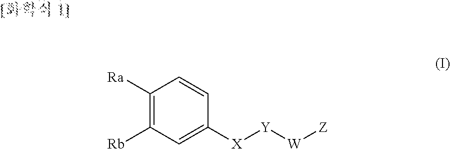

in formula (I),
wherein Ra and Rb are independently H or —O—CH₂—R1,
R1 is C6-C9 aryl optionally substituted with one or more halos,
X is —O—CH₂—CH(OH)—CH₂—NH— or —CH₂—NH—;
Y is —(CH₂CH₂—O)ₙ₁—, —(CH₂)ₙ₂—O—, n1 and n2 are independently an integer of 1 to 6;
W is —(CH₂)ₙ₃—NH—C(═O)—, —(CH₂)ₙ₄—NH—C(═O)—(CH₂)ₙ₃—O—, —CH₂—CH₂—O—, —CH₂—C(═O)—, n3, n4 and n5 are independently an integer of 1 to 7, preferably, —CH₂CH₂—NH—C(═O)—, Z is

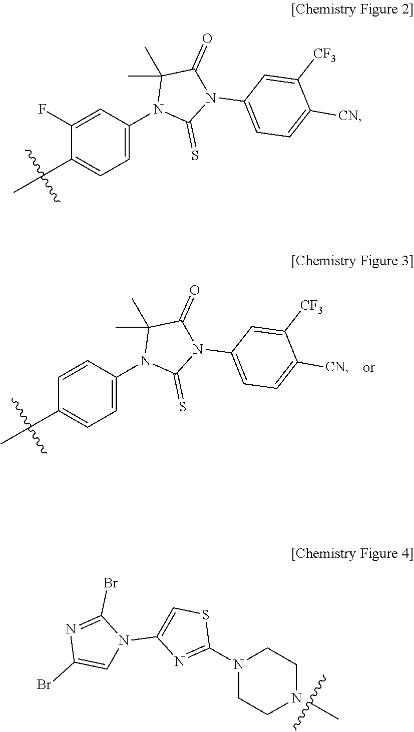

In certain embodiment, Ra and Rb are independently H or benzyloxy optionally substituted with one or two halogens.

In certain further embodiment, Ra and Rb are H or 4-fluorobenzyloxy.

In certain embodiment, Y is —(CH₂CH₂—O)ₙ₁—, —(CH₂)ₙ₂—O—, n1 and n2 are independently an integer of 1 to 6;

In certain embodiment, W is —CH₂CH₂—NH—C(═O)—, —CH₂CH₂—O—, —CH₂C(═O)—, —(CH₂)₆—NH—C(═O)—, —(CH₂)₂—NH—C(═O)—(CH₂)₇—O—.

Representative compounds of Formula (I) are listed below:
(R)—N-(2-(2-(2-((3-(3,4-bis((4-fluorobenzyl)oxy)phenoxy)-2-hydroxypropyl)amino)ethoxy)ethoxy)ethyl)-4-(3-(4-cyano-3-(trifluoromethyl)phenyl)-5,5-dimethyl-4-oxo-2-thioxoimidazolidin-1-yl)-2-fluorobenzamide;
(R)—N-(18-(3,4-bis((4-fluorobenzyl)oxy)phenoxy)-17-hydroxy-3,6,9,12-tetraoxa-15-azaoctadecyl)-4-(3-(4-cyano-3-(trifluoromethyl)phenyl)-5,5-dimethyl-4-oxo-2-thioxoimidazolidin-1-yl)-2-fluorobenzamide;
(R)—N-(24-(3,4-bis((4-fluorobenzyl)oxy)phenoxy)-23-hydroxy-3,6,9,12,15,18-hexaoxa-21-azatetracosyl)-4-(3-(4-cyno-3-(trifluoromethyl)phenyl)-5,5-dimethyl-4-oxo-2-thioxoimidazolidin-1-yl)-2-fluorobenzamide;
(R)-18-(3,4-bis((4-fluorobenzyl)oxy)phenoxy)-1-(4-(4-(2,4-dibromo-1H-imidazol-1-yl)thiazol-2-yl)piperazin-1-yl)-3,6,9,12-tetraoxa-15-azaoctadecan-17-ol;
6(R)-18-(3,4-bis((4-fluorobenzyl)oxy)phenoxy)-1-(4-(4-(2,4-dibromo-1H-imidazol-1-yl)thiazol-2-yl)piperazin-1-yl)-17-hydroxy-3,6,9,12-tetraoxa-15-azaoctadecan-1-one;
(R)-4-(3-(4-cyano-3-(trifluoromethyl)phenyl)-5,5-dimethyl-4-oxo-2-thioxoimidazolidin-1-yl)-2-fluoro-N-(18-(3-((4-fluorobenzyl)oxy)phenoxy)-17-hydroxy-3,6,9,12-tetraoxa-15-azaoctadecyl)benzamide;
(R)-4-(3-(4-cyano-3-(trifluoromethyl)phenyl)-5,5-dimethyl-4-oxo-2-thioxoimidazolidin-1-yl)-2-fluoro-N-(6-((5-((3-(3-((4-fluorobenzyl)oxy)phenoxy)-2-hydroxypropyl)amino)pentyl)oxy)hexyl)benzamide;
8-(4-(3-(4-cyano-3-(trifluoromethyl)phenyl)-5,5-dimethyl-4-oxo-2-thioxoimidazolidin-1-yl)phenoxy)-N-(2-(2-(2-((3-((4-fluorobenzyloxy)benzyl)amino)ethoxy)ethoxy)ethyl)octanamide; and
4-(3-(4-((1-(3-((4-fluorobenzyl)oxy)phenyl-5,8,11,14,17-pentaoxa-2-azanonadecan-19-yl)oxy)phenyl)-4,4-dimethyl-5-oxo-2-thioxoimidazolidin-1-yl)-2-(trifluoromethyl)benzonitrile.

Single stereochemical isomers, enantiomers, diastereomers, and pharmaceutically acceptable suits of the above exemplified compounds are also within the scope of the present invention. Pharmaceutically acceptable salts may be, for example, derived from suitable inorganic and organic acids and bases.

Acid addition salts can be prepared by reacting the purified compound in its free-based form, if possible, with a suitable organic or inorganic acid and isolating the salt thus formed. Examples of pharmaceutically acceptable acid addition salts include, without limitations, salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid.

Base addition salts can be prepared by reacting the purified compound in its acid form with a suitable organic or inorganic base and isolating the salt thus formed. Such salts include, without limitations, alkali metal (e.g., sodium, lithium, and potassium), alkaline earth metal (e.g., magnesium and calcium), ammonium and N—(C₁₋₄alkyl), salts.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethane-sulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, and valerate salts.

In addition, the compounds represented by Formula (I) according to the present invention include, but are not limited thereto, not only pharmaceutically acceptable salts thereof, but also all solvates or hydrates and all possible stereoisomers that can be prepared therefrom.

All stereoisomers of the present compounds (e.g., those which may exist due to asymmetric carbons on various substituents), including enantiomeric forms and diastereomeric forms, are contemplated within the scope of this invention. Individual stereoisomers of the compounds of the present invention may, for example, be substantially free of other isomers (e.g., as a pure or substantially pure optical isomer having a specified activity), or may be admixed, for example, as racemates or with all other, or other selected, stereoisomers. The chiral centers of the compounds of the present invention may have the S or R configuration as defined by the IUPAC 1974 Recommendations. The racemic forms can be analyzed by physical methods, such as fractional crystallization, separation or crystallization of diastereomeric derivatives or separation by chiral column chromatography. The individual optical isomers can be obtained from the racemates by any suitable method, including without limitation, salt formation with an optically active acid followed by crystallization.

Where the compound contains, for example, a keto or oxime group or an aromatic moiety, tautomeric isomerism (‘tautomerism’) can occur. It follows that a single compound may exhibit more than one type of isomerism.

Included within the scope of the claimed compounds of the present invention are all stereoisomers, geometric isomers and tautomeric forms of the compounds of the invention, including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

The compounds of the present invention may be administered as prodrugs. Thus certain derivatives of compounds of the invention which may have little or no pharmacological activity themselves can, when administered into or onto the body, be convened into compounds of formula I (or other formulae disclosed herein) having the desired activity, for example, by hydrolytic cleavage. Such derivatives are referred to as on may be admProdrugs can, for example, be produced by replacing appropriate functionalities present in the compounds of the invention with certain moieties known to those skilled in the art as ‘pro-moieties’ as described.

Some examples of such prodrugs include:
where the compound contains a carboxylic acid functionality (—COOH), an ester thereof, for example, replacement of the hydrogen with C1-C6 alkyl;
where the compound contains an alcohol functionality (ality (—COOH), an ester thereof, for example, replacement of the hydrog1-C6 alkanoyloxymethyl (cC1-C6 acyloxymethyl); and
where the compound contains a primary or secondary amino functionality (est2 or —NHR where R is not H), an amide thereof, for example, replacement of one or both hydrogens with (C1-C10)alkanoyl (—C1-C10 acyl).

The solvate and stereoisomer of the compound represented by Formula (I) may be prepared from the compound represented by Formula (I) using methods known in the art.

Furthermore, the compounds represented by Formula (I) according to the present invention may be prepared either in a crystalline form or in a non-crystalline form. When the compound is prepared in a crystalline form, it may be optionally hydrated or solvated. In the present invention, the compound of Formula (I) may not only include a stoichiometric hydrate, but also include a compound containing various amounts of water. The solvate of the compound of Formula (I) according to the present invention includes both stoichiometric solvates and non-stoichiometric solvates.

The compounds of the present invention may be synthesized by methods known in the art or by methods illustrated in Examples 1-9 below.

In one embodiment, the present invention relates to a method for treating AR related disease in a subject in need thereof, comprising administering to said subject a therapeutically effective amount of a compound of Formula (I) or a pharmaceutically acceptable salt thereof. In specific embodiment, the AR-mediated disease is a prostate cancer, specifically castration resistant prostate cancer (CRPC). The invention further relates to therapeutic methods and uses comprising administering the compounds of the invention, or pharmaceutically acceptable salts thereof, alone or in combination with other therapeutic or palliative agents.

A further embodiment of the invention relates to a compound of the invention for use as a medicament, and in particular for use in the treatment of diseases where the inhibition of AR signaling through sequestration and degradation of AR may induce benefit, such as prostate cancer. A still further embodiment of the present invention relates to the use of the compounds of the invention, or pharmaceutically acceptable salts thereof, for the manufacture of a drug having an AR degradative activity for the treatment of AR mediated diseases and/or conditions, in particular the diseases and/or conditions mentioned above.

The term “therapeutically effective amount” refers to that amount of a compound being administered which will relieve to some extent one or more of the symptoms of the disorder being treated. Regarding the treatment of cancer, a therapeutically effective amount refers to that amount which has the effect of reducing the size of the tumor, inhibiting (i.e., slowing or stopping) tumor metastases, inhibiting (i.e. slowing or stopping) tumor growth or tumor invasiveness, and/or relieving to some extent one or more signs or symptoms related to the cancer.

A therapeutically effective amount can be readily determined by the attending diagnostician, as one skilled in the art, by the use of conventional techniques and by observing results obtained under analogous circumstances. In determining the therapeutically effective amount, the dose, a number of factors are considered by the attending diagnostician, including, but not limited to: the species of mammal; its size, age, and general health; the specific disease involved; the degree of involvement or the severity of the disease; the response of the individual patient; the particular compound administered; the mode of administration; the bioavailability characteristic of the preparation administered; the dose regimen selected; the use of concomitant medication; and other relevant circumstances.

The term “treating”, as used herein, unless otherwise indicated, means reversing, alleviating, inhibiting the progress of, or preventing the disorder or condition to which such term applies, or one or more symptoms of such disorder or condition. The term “treatment” also refers to the act of treating as “treating” is defined immediately above. The term “treating” also includes adjuvant treatment of a mammal.

Administration of the compounds of the invention may be effected by any method that enables delivery of the compounds to the site of action. These methods include oral routes, intraduodenal routes, parenteral injection (including intravenous, subcutaneous, intramuscular, intravascular or infusion), topical, and rectal administration.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage.

Dosage unit form, as used herein, refers to physically discrete units suited as unitary dosages for the mammalian mammals to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Appropriate dosages may vary with the type and severity of the condition to be treated and may include single or multiple doses. An attending diagnostician understands that for any particular mammal, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

Useful dosages of the compounds of the invention can be determined by comparing their in vitro activity, and in vivo activity in animal models. The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

Also provided herein, in other aspects, are pharmaceutical compositions comprising compounds of the Formula (I), as active ingredients, and pharmaceutically acceptable excipients, in one embodiment, the pharmaceutical composition is for treating AR-related disease such as prostate cancer. In another embodiment, the pharmaceutical composition is for degradation of AR.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions suitable for the delivery of compounds of the invention and methods for their preparation will be readily apparent to those skilled in the art.

The compounds of the invention may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid.

Examples of carriers, excipients and diluents that can be included in the composition, may include lactose, dextrose, sucrose, sorbitol, mannitol, xylitol, erythritol, maltitol, starch, arabic gum, alginate, gelatin, calcium phosphate, calcium silicate, cellulose, methylcellulose, microcrystalline cellulose, polyvinyl pyrrolidone, water, methyl hydroxybenzoate, propyl hydroxybenzoate, talc, magnesium stearate, and mineral oil, but are not limited thereto. When formulated into a preparation, a diluting agent or an excipient, such as commonly-used fillers, stabilizing agents, binding agents, disintegrating agents, and surfactants can be used. Solid preparations for oral administration include tablets, pills, powders, granules, capsules, and the like, and these solid preparations may be prepared by mixing the compound of the present invention with at least one excipient, for example, starch, microcrystalline cellulose, sucrose, lactose, low-substituted hydroxypropyl cellulose, hypromellose or the like. In addition to the simple excipient, a lubricant such as magnesium stearate and talc are also used. Liquid preparations for oral administration include a suspension, a liquid for internal use, an emulsion, a syrup, etc. In addition to a commonly used simple diluent such as water and liquid paraffin, various excipients such as a humectant, a sweetener, an aromatic, a preservative, etc. may also be contained. Formulations for parenteral administration include a sterilized aqueous solution, a non-aqueous solution, a suspension, an emulsion, a lyophilized formulation and a suppository. The non-aqueous solution or suspension may contain propylene glycol, polyethylene glycol, a vegetable oil such as olive oil, an injectable ester such as ethyl oleate, etc. As a base of the suppository, witepsol, macrogol, tween 61, cocoa butter, laurin butter, glycerogelatin, etc. may be used. In order to formulate the formulation for parenteral administration, the compound of Formula I or a pharmaceutically acceptable salt thereof may be mixed in water together with sterilized and/or contain adjuvants such as preservatives, stabilizers, auxiliary agents such as wettable powder or emulsifying accelerators, salt for controlling osmotic pressure and/or buffers and the like, and other therapeutically useful substances, to prepare a solution or suspension, which is then manufactured in the form of an ampoule or vial unit administration.

The term “combination therapy” refers to the administration of a compound of the invention together with at least one additional pharmaceutical or medicinal agent, either sequentially or simultaneously. Combination therapy encompasses the use of the compounds of the present invention and other therapeutic agents either in discreet dosage forms or in the same pharmaceutical formulation. The compounds of the invention may be used in combination (administered simultaneously, sequentially, or separately) with one or more therapeutic agents.

In one embodiment of the present invention the anti-cancer agent used in conjunction with a compound of the invention and pharmaceutical compositions described herein is an antiangiogenesis agent (e.g., an agent that stops tumors from developing new blood vessels). Examples of anti-angiogenesis agents include for example VEGF inhibitors, VEGFR inhibitors, TIE-2 inhibitors, PDGFR inhibitors, angiopoietin inhibitors, PKCI3 inhibitors, CQX-2 (cyclooxygenase II) inhibitors, integrins (alpha-v/beta-3), MMP-2 (matrix-metalloproteinase 2) inhibitors, and MMP-9 (matriximetalloproteinase 9) inhibitors. Preferred anti-angiogenesis agents include sunitinib (SutenFM), bevacizumab (Avastin™), and axitinib (AG 13736).

### MODE FOR THE INVENTION

### EXAMPLES

The present invention is further exemplified by the following examples that illustrate the preparation of compounds of Formula (I) according to the invention. The examples are for illustrative purpose only and are not intended, nor should they be construed as limiting the invention in any manner. Those skilled in the art will appreciate that variations and modifications can be made without changing the scope of the invention.

The compounds of Examples 1 to 9 have been prepared via below preparation method.

| Example | ID | Name |
|---|---|---|
| Example 1 | Compound | (R)-N-(2-(2-(2-((3-(3,4-bis((4-fluorobenzyl)oxy)phenoxy)-2- |
| | A | hydroxypropyl)amino)ethoxy)ethyl)-4-(3-(4-cyano-3- |
| | | (trifluoromethyl)phenyl)-5,5-dimethyl-4-oxo-2- |
| | | thioxoimidazolidin-1-yl)-2-fluorobenzamide |
| Example 2 | Compound | (R)-N-(18-(3,4-bis((4-fluorobenzyl)oxy)phenoxy)-17-hydroxy- |
| | B | 3,6,9,12-tetraoxa-15-azaoctadecyl)-4-(3-(4-cyano-3- |
| | | (trifluoromethyl)phenyl)-5,5-dimethyl-4-oxo-2- |
| | | thioxoimidazolidin-1-yl)-2-fluorobenzamide |
| Example 3 | Compound | (R)-N-(24-(3,4-bis((4-fluorobenzyl)oxy)phenoxy)-23-hydroxy- |
| | C | 3,6,9,12,15,18-hexaoxa-21-azatetracosyl)-4-(3-(4-cyano-3- |
| | | (trifluoromethyl)phenyl)-5,5-dimethyl-4-oxo-2- |
| | | thioxoimidazolidin-1-yl)-2-fluorobenzamide |
| Example 4 | Compound | (R)-18-(3,4-bis((4-fluorobenzyl)oxy)phenoxy)-1-(4-(4-(2,4- |
| | D | dibromo-1H-imidazol-1-yl)thiazol-2-yl)piperazin-1-yl)-3,6,9,12- |
| | | tetraoxa-15-azaoctadecan-17-ol |
| Example 5 | Compound | (R)-18-(3,4-bis((4-fluorobenzyl)oxy)phenoxy)-1-(4-(4-(2,4- |
| | E | dibromo-1H-imidazol-1-yl)thiazol-2-yl)piperazin-1-yl)-17- |
| | | hydroxy-3,6,9,12-tetraoxa-15-azaoctadecan-1-one |
| Example 6 | Compound | (R)-4-(3-(4-cyano-3-(trifluoromethy)phenyl)-5,5-dimethyl-4- |
| | F | oxo-2-thioxoimidazolidin-1-yl)-2-fluoro-N-(18-(3-((4- |
| | | fluorobenzyl)oxy)phenoxy)-17-hydroxy-3,6,9,12-tetraoxa-15- |
| | | azaoctadecyl)benzamide |
| Example 7 | Compound | (R)-4-(3-(4-cyano-3-(trifluoromethy)phenyl)-5,5-dimethyl-4- |
| | G | oxo-2-thioxoimidazolidin-1-yl)-2-fluoro-N-(6-((5-((3-(3-((4- |
| | | fluorobenzyl)oxy)phenoxy)-2- |
| | | hydroxypropyl)amino)pentyl)oxy)hexyl)benzamide |
| Example 8 | Compound | 8-(4-(3-(4-cyano-3-(trifluoromethyl)phenyl)-5,5-dimethyl-4- |
| | H | oxo-2-thioxoimidazolidin-1-yl)phenoxy)-N-(2-(2-(2-((3-((4- |
| | | fluorobenzyl)oxy)benzyl)amino)ethoxy)ethoxy)ethyl)octanamide |
| Example 9 | Compound | 4-(3-(4-((1-(3-((4-fluorobenzyl)oxy)phenyl)-5,8,11,14,17- |
| | I | pentaoxa-2-azanonadecan-19-yl)oxy)phenyl)-4,4-dimethyl-5- |
| | | oxo-2-thioxoimidazolidin-1-yl)-2-(trifluoromethyl)benzonitrile |

1H NMR spectra were recorded on Bruker Avance III 400 MHz and Agilent VNMRS 600 MHz and TMS was used as an internal standard LCMS was taken on a quadrupole Mass Spectrometer like below;

SHIMADZU LC/MS-2020 (Column: C18 (4.6>5.0 mm, 5 μm) operating in ES (+) or (−) ionization mode; T=40° C.; flow rate=1.5 mL/min; detected wavelength: 190-800 nm.

Agilent 1260HPLC and 6120MSD (Column: C18 (50>4.6 mm, 5 μm) operating in ES (+) or (−) ionization mode; T=30° C.; flow rate=1.5 mL/min; detected wavelength: 220 nm, 254 nm

### Example 1. Preparation of Compound A ((R)—N-(2-(2-(2-((3-(3,4-bis((4-fluorobenzyl)oxy)phenoxy)-2-hydroxypropyl)amino)ethoxy)ethoxy)ethyl)-4(3-(4-cyano-3-(trifluoromethyl)phenyl)-5,5-dimethyl-4-oxo-2-thioxoimidazolidin-1-yl)-2-fluorobenzamide

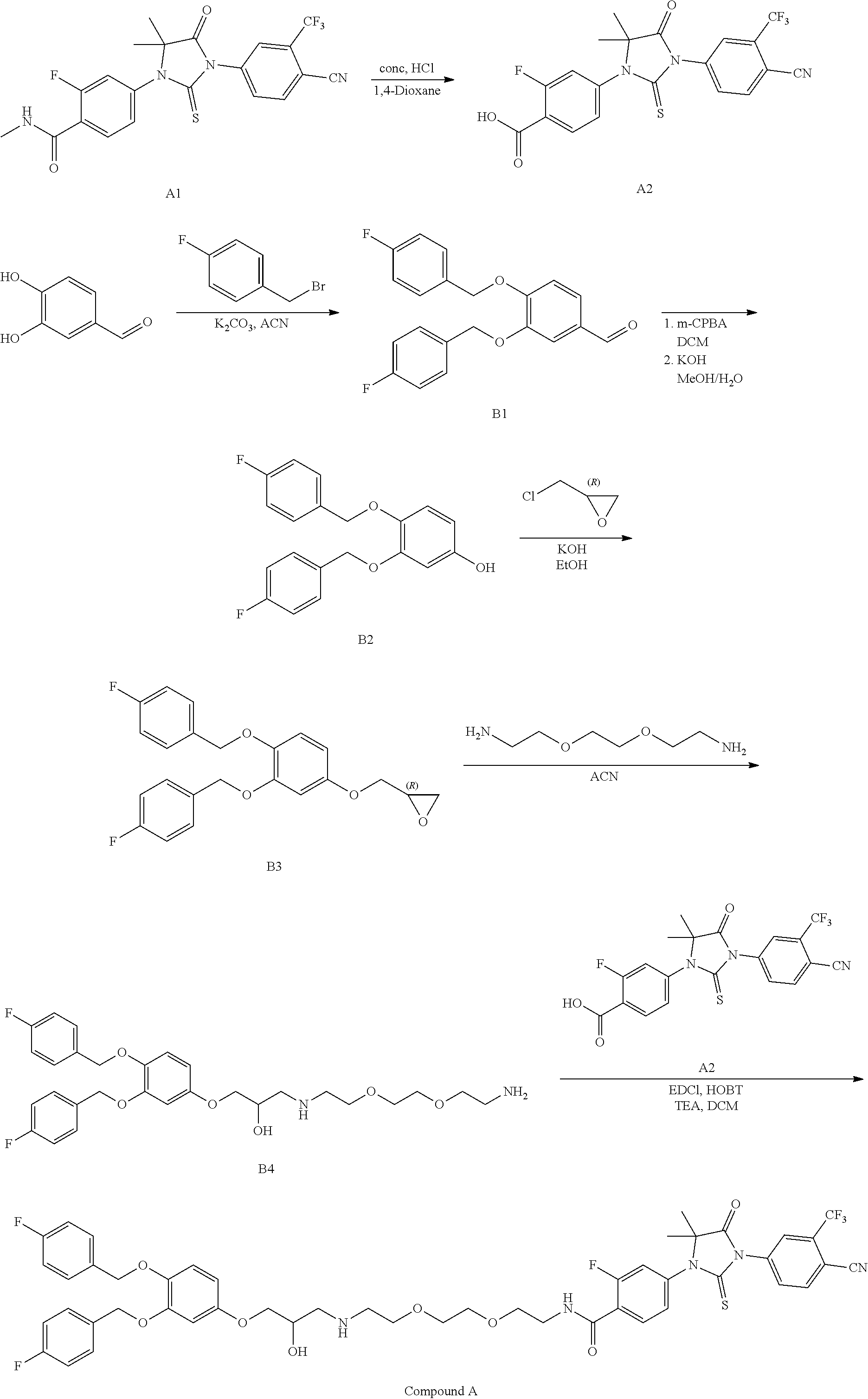

### Step 1) Synthesis A2

To a solution of A1 (15 g, 32.3 mmol) in dioxane (150 mL) was added con. HCl (150 mL), then the mixture was heated to reflux and stirred for 3 days. Water was added and the mixture was extracted with ethyl acetate (EA). The organic phase was dried over Na₂SO₄ and concentrated. The residue was purified by column (PE:EA=5:1˜2:1) to afford A2 (9.0 g, 20.0 mmol, 61.9% yield) as a pale yellow solid.

¹HNMR (CDCl3, 400 MHz): δ 8.25-8.21 (m, 1H), 8.01-7.95 (m, 2H), 7.85-7.82 (m, 1H), 7.27-7.20 (m, 21H), 1.64 (s, 6H).

### Step 2) Synthesis of B1

To a solution of 3,4-dihydroxybenzaldehyde (100 g, 724.6 mmol) in acetonitrile (ACN, 1000 mL) were added 1-(bromomethyl)-4-fluorobenzene (301.3 g, 1.59 mol) and K₂CO₃ (300 g, 2.17 mol). The mixture was stirred at 80° C. for 16 hrs. Then the reaction was concentrated, the residue was purified by silica gel, eluted with EA/PE (1:15˜1:8) to afford B1 (3,4-bis((4-fluorobenzyl)oxy)benzaldehyde, 187 g) as a white solid.

¹HNMR (DMSO-d6, 400 MHz): δ 9.83 (s, 1H), 7.55-7.48 (m, 6H), 7.30-7.20 (m, 5H), 5.25 (s, 2H), 5.19 (s, 2H).

### Step 3) Synthesis of B2

To a solution of B1 (187 g, 526.7 mmol) in dichloromethane (DCM, 2000 mL) was added 3-Chloroperbenzoic acid (m-CPBA, 126 g, 730.4 mmol). The mixture was stirred at room temperature for 16 hrs. Then the reaction was washed with saturated sodium bicarbonate solution, concentrated under vacuum. Then the crude product is added to methanol (1500 mL) and water (200 mL) was added KOH (58.9 g, 1.05 mol). The mixture was stirred at room temperature for 3 hrs. Then the reaction was filtered and the solid to dryness under vacuum. The crude compound was purified by silica gel, eluted with EA/PE (1:15˜1:5) to afford B2 (3,4-bis((4-fluorobenzyl)oxy)phenol, 151 g) as an off-white solid.

¹HNMR (DMSO-d6, 400 MHz): δ 7.49-7.41 (m, 4H), 7.24-7.15 (m, 4H), 6.82 (d, J=8.4 Hz, 1H), 6.49 (s, 1H), 6.25 (d/d, 1H), 5.04 (s, 2H), 5.00 (s, 2H).

### Step 4) Synthesis of B3

To a solution of B2 (45.8 g, 134 mmol) in ethanol (EtOH, 500 mL) were added water (25 mL) and KOH (17.2 g, 307 mmol). Then (R)-2-(chloromethyl)oxirane (37 g, 400 mmol) was added to the reaction. The resulting mixture was stirred at room temperature for 16 hrs. Then the reaction was quenched by addition water, extracted with EA. The organic layer was washed with brine, dried over Na₂SO₄, filtered and concentrated. The residue was purified by silica gel, eluted with EA/PE (1:15˜1:10) to afford B3 ((R)-2-(3,4-bis((4-fluorobenzyl)oxy)phenoxy)methyl)oxirane, 26 g) as a white solid.

¹HNMR (DMSO-d6, 400 MHz): δ 7.51-7.42 (m, 4H), 7.25-7.16 (m, 4H), 6.94 (d, J=8.4 Hz, 1H), 6.73 (s, 1H), 6.46 (d/d, 1H), 5.10 (s, 2H), 5.00 (s, 2H), 4.24 (d/d, 1H), 3.77-3.73 (m, 1H), 3.30-3.28 (m, 1H), 2.84-2.82 (m, 1H), 2.69-2.67 (m, 1H).

### Step 5) Synthesis of B4

To a solution of B3 (200 mg, 0.502 mmol) in ACN (10 mL) was added 2,2′(ethane-1,2-diylbis(oxy))diethanamine (111 mg, 0.753 mmol). The solution was stirred at 70° C. for 16 hrs. Then the solution was concentrated. The residue was purified by pre-HPLC to afford B4 ((R)-1-((2-(2-(2-aminoethoxy)ethoxy)ethyl)amino)-3-(3,4-bis((4-fluorobenzyl)oxy)phenoxy)propan-2-ol, 60 mg) as a yellow solid.

₁H-NMR (DMSO-d6, 400 MHz): δ 7.49-7.42 (m, 4H), 7.24-7.18 (m, 4H), 6.92 (d, J=Hz, 1H), 6.67 (s, 1H), 6.48-6.46 (m, 1H), 5.09 (s, 2H), 4.99 (s, 2H), 3.83 (m, 3H), 3.50-3.35 (m, 8H), 2.67-2.62 (m, 6H).

### Step 6) Synthesis of Compound A ((R)—N-(2-(2-(2-((3-(3,4-bis((4-fluorobenzyl)oxy)phenoxy)-2-hydroxypropyl)amino)ethoxy)ethoxy)ethyl)-4-(3-(4-cyano-3-(trifluoromethyl)phenyl)-5,5-dimethyl-4-oxo-2-thioxoimidazolidin-1-yl)-2-fluorobenzamide)

To a solution of compound B4 (60 mg, 0.1 mmol) in DCM (10 mL) was added HOBT (16.3 mg, 0.12 mmol), EDCI (23 mg, 0.12 mmol) and Et3N (50.5 mg, 0.5 mmol). The solution of 4-phenylbutanoic acid (45.2 mg, 0.1 mmol) in DCM (5 mL) was added at 0° C. The solution was concentrated and the residue was purified by pre-HPLC to give Compound A ((R)—N-(2-(2-(2-((3-(3,4-bis((4-(fluorobenzyl)oxy)phenoxy)-2-hydroxypropyl)amino)ethoxy)ethoxy)ethyl)-4-(3-(4-cyano-3-(trifluoromethyl)phenyl)-5,5-dimethyl-4-oxo-2-thioxoimidazolidin-1-yl)-2-fluorobenzamide, 17 mg) as white solid.

₁H-NMR (DMSO-d6, 400 MHz): δ 8.38 (d, J=8.4 Hz, 1H), 8.28 (d, J=6 Hz, 2H), 8.07 (d, J=8 Hz, 1H), 7.77 (t, J=8 Hz, 1H), 7.49-7.42 (m, 5H), 7.33 (d/d, J=8.4 Hz, 1H), 7.23-7.15 (m, 4H), 6.93 (d, J=8.8 Hz 1H), 6.66 (d, J=2-8 Hz, 1H), 6.43 (d/d, J=8.8 Hz and 2.8 Hz, 1H), 5.08 (s, 2H), 4.99 (s, 2H), 3.85-3.82 (m, 3H), 3.45-3.42 (m, 10H), 2.83-2.79 (m, 3H), 2.72-2.69 (m, 1H), 1.53 (s, 6H)

LCMS; Mass Calcd.: 979.9; MS Found: 980.8 [MS+1].

### Example 2. Preparation of Compound B (R)—N-(18-(3,4-bis((4-fluorobenzyl)oxy)phenoxy)-17-hydroxy-3,6,9,12-tetraoxa-15-azaoctadecyl)-4-(3-(4-cyano-3-(trifluoromethyl)phenyl)-5,5-dimethyl-4-oxo-2-thioxoimidazolidin-1-yl)-2-fluorobenzamide

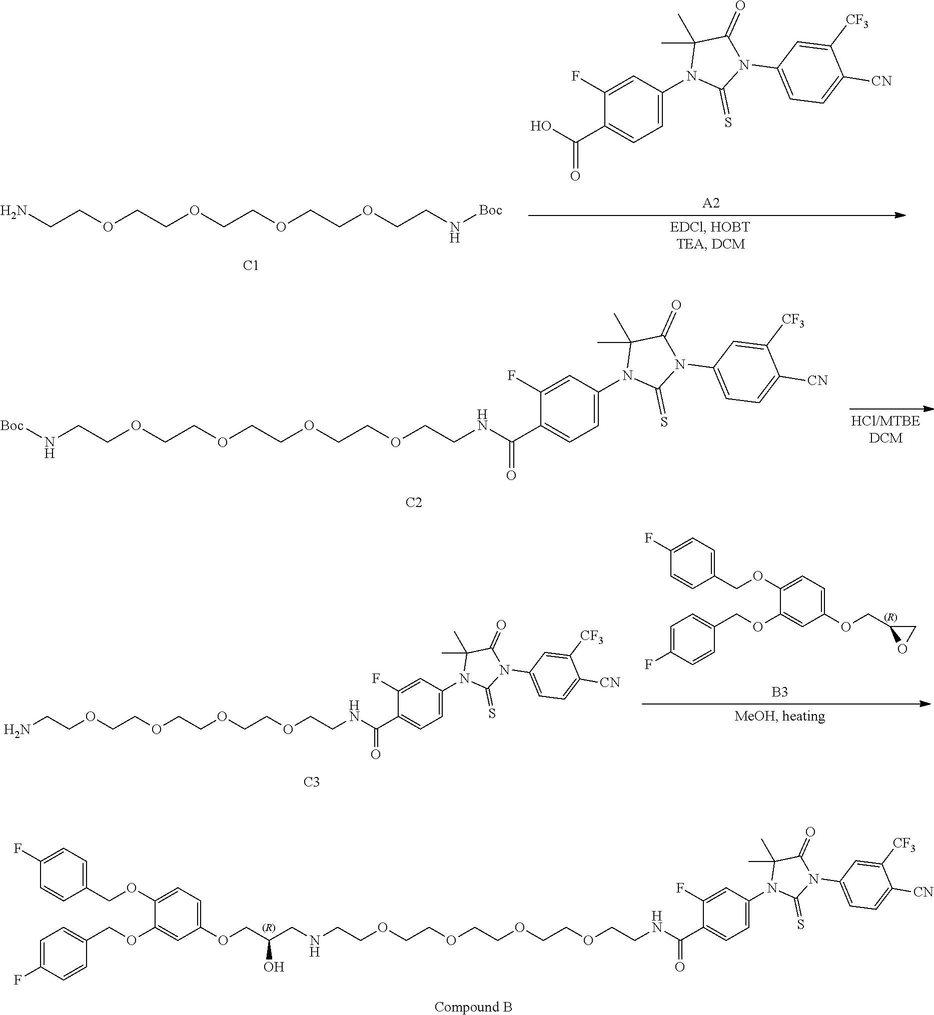

### Step 1) Synthesis of C2

To a solution of C1 (tert-butyl (14-amino-3,6,9,12-tetraoxatetradecyl)carbamate, 4.0 g, 11.9 mmol) in DCM (100 mL) were added A2 (5.37 g, 11.9 mmol), 1-ethyl-3-(3-dimethylaminopropyl)carbodiimide (EDCI, 3.41 g, 17.8 mmol), 1-hydroxybenzotriazole (HOBT, 2.40 g, 17.8 mmol) and trimethylamine (TEA, 2.40 g, 23.8 mmol), then the mixture was stirred at room temperature overnight. The mixture was washed with water and brine.

The organic phase was dried over Na2SO4 and concentrated. The residue was purified by column (DCM:MeOH=50:1˜20:1) to afford C-2 (7.5 g, 9.75 mmol, 82.0% yield) as colorless oil.

₁H-NMR (CDCl₃, 400 MHz): δ 8.27-8.23 (m, 1H), 8.02-7.97 (m, 2H) 7.86-7.83 (m, 1H), 7.28-7.16 (m, 3H), 5.07 (br s, 1H), 3.79-3.60 (m, 16H), 3.56-3.53 (m, 2H), 3.32 (m, 2H), 1.66 (s, 6H), 1.45 (s, 9H).

### Step 2) Synthesis of C3

To a solution of C2 (7.5 g, 9.75 mmol) in DCM (75 mL) was added HCl/MTBE (75 mL, 8 mol/L), then the mixture was stirred at room temperature for 4 hours. The mixture was concentrated and dissolved in DCM, then washed with saturated aqueous Na2CO3 and brine. The organic phase was dried over Na2SO4 and concentrated to afford C3 (6.0 g, 8.97 mmol, 92.0% yield) as yellow oil.

₁H-NMR (CDCl₃, 400 MHz): δ 8.23-8.20 (m, 1H), 8.02-7.97 (m, 2H), 7.86-7.84 (m, 1H), 7.48 (br s, 1H), 7.28-7.15 (m, 2H), 3.79-3.62 (m, 16H), 3.52-3.50 (m, 2H), 2.83 (t, 2H), 1.63 (s, 6H).

### Step 3) Synthesis of Compound B ((R)—N-(18-(3,4-bis((4-fluorobenzyl)oxy)phenoxy)-17-hydroxy-3,6,9,12-tetraoxa-15-azaoctadecyl)-4-(3-(4-cyano-3-(trifluoromethyl)phenyl-5,5-dimethyl-4-oxo-2-thioxoimidazolidin-1-yl)-2-fluorobenzamide)

To a solution of C3 (3.0 g, 4.48 mmol) in MeOH (6 mL) and acetonitrile (6 mL) was added (R)-2-((3,4-bis(4-fluorobenzyloxy)phenoxy)methyl)oxirane (1.78 g, 4.48 mmol), then the mixture was stirred at 65° C. overnight. The mixture was concentrated and purified by column (DCM:MeOH=50:1-20:1) to afford compound Compound B ((R)—N-(18-(3,4-bis((4-fluorobenzyl)oxy)phenoxy)-17-hydroxy-3,6,9,12-tetraoxa-15-azaoctadecyl)-4-(3-(4-cyano-3-(trifluoromethyl)phenyl)-5,5-dimethyl-4-oxo-2-thioxoimidazolidin-1-yl)-2-fluorobenzamide, 1.1 g, 1.03 mmol, 23.0% yield) as a pale yellow solid.

1H-NMR (CD3OD, 400 MHz): δ 8.22 (t, J=8.4 Hz, 1H), 7.97-8.03 (m, 2H), 7.84 (dd, J1=8.0 Hz, J2=2.0 Hz, 1H), 7.30-7.42 (m, 5H), 7.14-7.25 (m, 2H), 7.01-7.08 (m, 4H), 6.85 (d, J=8.8 Hz, 1H), 6.60 (d, J=2.8 Hz, 1H), 6.41 (dd, J1=8.8 Hz, J2=2.8 Hz, 1H), 5.07 (s, 2H), 5.02 (s, 2H), 4.05-4.08 (m, 1H), 3.90 (d, J=9.2 Hz, 2H), 3.62-3.71 (m, 18H), 2.76-2.93 (m, 5H), 1.62 (s, 6H).

LCMS: Mass Calcd.: 1068, MS Found: 1068 [MS+1].

### Example 3. Preparation of Compound C ((R)—N-(24-(3,4-bis((4-

### fluorobenzyl)oxy)phenoxy)-23-hydroxy-3,6,9,12,15,18-hexaoxa-21-azatetracosyl)-4-(3-(4-cyano-3-(trifluormethyl)phenyl)-5,5-dimethyl-4-oxo-2-thioxoimidazolidin-1-yl)-2-fluorobenzamide)

Compound C was synthesized as white solid in the sane manner as in Example 2 by using tert-butyl (20-amino-3,6,9,12,15,18-hexaoxaicosyl)carbamate as a starting material instead of C1.

1H-NMR (CD3OD, 400 MHz): δ 8.24 (t, J=8.4 Hz, 1H), 7.98-8.02 (m, 2H), 7.85 (dd, J1=

8.4 Hz, J2=2.0 Hz, 1H), 7.36-7.43 (m, 4H), 7.16-7.26 (m, 3H), 7.01-7.09 (m, 4H), 6.85 (d, J=8.8 Hz, 1H), 6.60 (d, J=2.8 Hz, 1H), 6.41 (dd, J1=8.8 Hz, J2=2.8 Hz, 1H), 5.07 (s, 2H), 5.02 (s, 2H), 4.09-4.13 (m, 1H), 3.91-3.94 (m, 2H), 3.65-3.73 (m, 25H), 2.81-2.99 (m, 5H), 1.63 (s, 6H).

LCMS; Mass Calcd.: 1156; MS Found: 1156 [MS+1].

### Example 4. Preparation of Compound D ((R)-18-(3,4-bis((4-fluorobenzyl)oxy)phenoxy)-1-(4-(4-(2,4-dibromo-1H-imidazol-1-yl)thiazol-2-yl)piperazin-1-yl)-3,6,9,12-tetraoxa-15-azaoctadecan-17-ol)

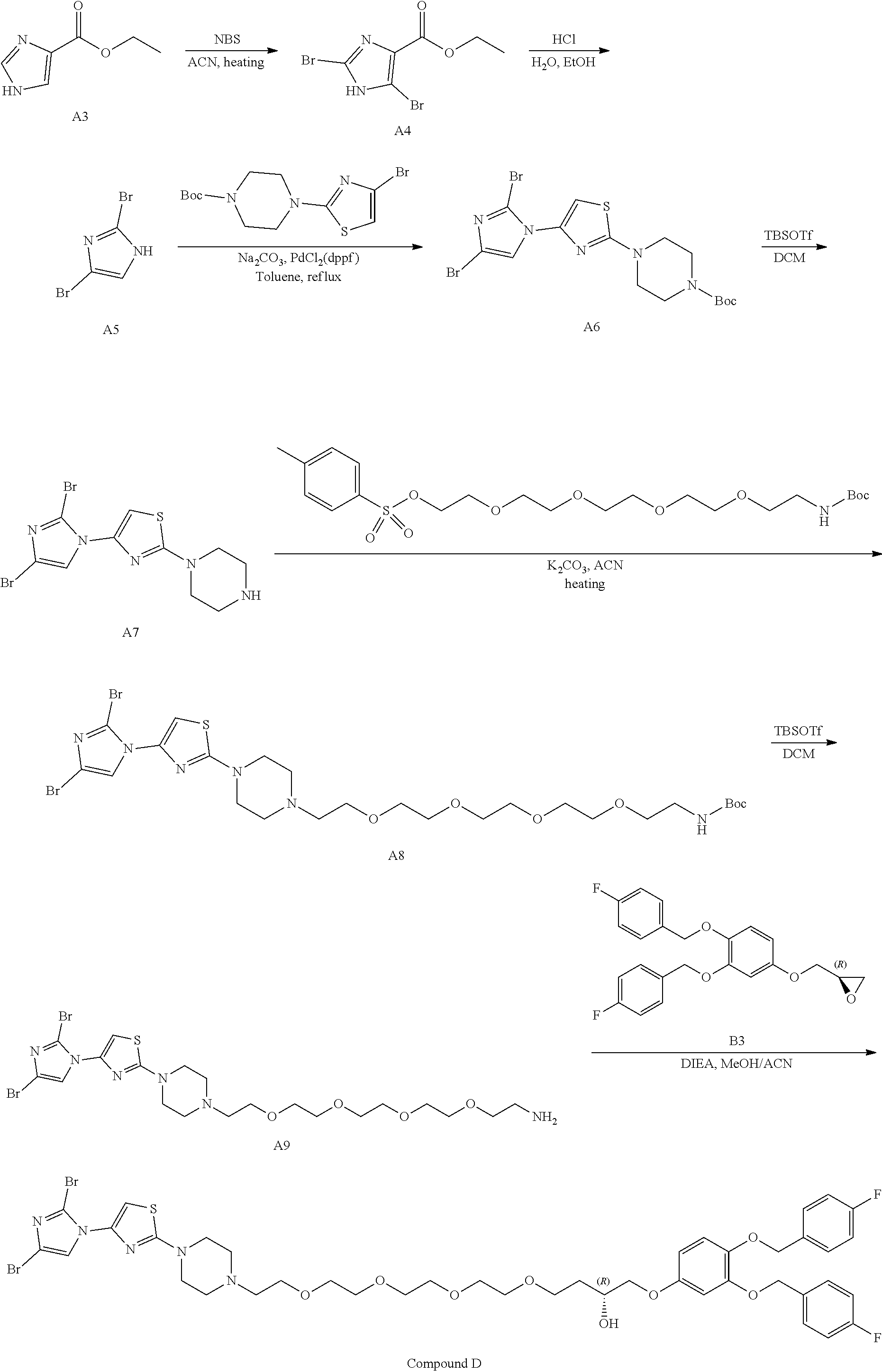

### Step 1) Synthesis of A4

A mixture of A3 (20.0 g, 143 mmol, 1.0 eq) and NBS (50.8 g, 285 mmol, 2.0 eq) in MeCN (300 mL) was stirred at 85° C. for 3 hrs. The mixture was poured into water and extracted with EA. The organic layer was washed with brine, dried over Na2SO4 and concentrated to give crude product, which was purified by flash column (PE/EA=5/1) to give A4 (20 g, yield 47%) as a white solid.

1H-NMR (CDCl3, 400 MHz): δ 11.13 (br s, 1H), 4.44 (q, J=7.2 Hz, 2H), 1.43 (t, J=7.2 Hz, 3H).

### Step 2) Synthesis of A5

A mixture of A4 (20.0 g, 67.1 mmol, 1.0 eq) in conc. HCl (200 ml). H2O (200 mL) and EtOH (40 mL) was stirred at reflux for 24 hrs. The mixture was concentrated The residue was added to water and the mixture was adjusted pH=8 with 4N aq. NaOH and extracted with EA. The organic layer was dried over Na2SO4 and concentrated to give crude product, which was purified by MPLC to give A5 (6 g, yield 40%) as a white solid. (TLC: PE/EA=2/1, Rf=0.5)

₁H-NMR (DMSO-d6, 400 MHz): δ 13.18 (br s, 1H), 7.41 (s, 1H).

### Step 3) Synthesis of A6

A mixture of A5 (8.1 g, 36 mmol, 1.0 eq), tert-butyl 4-(4-bromothiazol-2-yl) piperazine-1-carboxylate (12.5 g, 36 mmol, 1.0 eq), Na2CO3 (7.63 g, 72 mmol, 2.0 eq), PdCl2(dppf) (4.2 g) in toluene (250 mL) was stirred at reflux for 72 hrs. The mixture was concentrated. The residue was purified by flash column (PE/EA=10/1-5/1) to give crude compound 13, which was slurried in DCM for 2 hrs. The mixture was filtered and the filtrate was concentrated. Then the crude product was purified by prep-TLC to obtain A6 (500 mg, yield 2.7%) as a yellow solid. (TLC: PE/EA=2/1, Rf=0.6)

1H-NMR (CDCl3, 400 MHz): δ 7.41 (s, 1H), 6.70 (s, 1H), 3.59-3.61 (m, 4H), 3.49-3.51 (m, 4H), 1.51 (s, 9H).

### Step 4) Synthesis of A7

A mixture of A6 (500 mg, 1.01 mmol, 1.0 eq) and TBSOTf (260 mg, 1.01 mmol, 1.0 eq) in DCM (10 mL) was stirred at room temperature for 2 hrs. The mixture was quenched with NH₄Cl and adjusted to pH>7 with aq. NaHCO3. The mixture was extracted three times with DCM. The combined organic layers were dried over Na2SO4 and concentrate to give A7 (300 mg, crude) as a yellow solid. (TLC: DCM/MeOH=20/1, Rf=0.3)

1H-NMR (CDCl3, 400 MHz): δ 7.42 (s, 1H), 6.67 (s, 1H), 3.48-3.51 (m, 4H), 3.01-3.04 (m, 4H).

### Step 5) Synthesis of A8

A mixture of A7 (400 mg, 1.02 mmol, 1.0 eq), 2,2-dimethyl-4-oxo-3,8,11,14,17-pentaoxa-5-azanonadecan-19-yl 4-methylbenzenesulfonate (600 mg, 1.22 mmol, 1.0 eq) and K2CO3 (844 mg, 6.11 mmol, 6.0 eq) in acetonitrile (CH3CN, 10 mL) was stirred at 70° C. for 72 hrs. The mixture was concentrated. The residue was added to water and extracted three times with EA. The combined organic layers were dried over Na2SO4 and concentrated to obtain A8 (700 nig, crude) as yellow oil. (TLC: DCM/MeOH=10/1, Rf=0.5)

LCMS: Mass Calcd.: 712.50; MS Found: 713.4 [MS+1].

### Step 6) Synthesis of A9

To a mixture of A8 (700 mg, 0.98 mmol, 1.0 eq) in DCM (10 mL) was added TBSOTf (260 mg, 0.98 mmol, 1.0 eq) at 20° C. The mixture was stirred at 20° C. for 2 hrs. Then another batch of TBSOTf (260 mg, 0.98 mmol, 1.0 eq) was added. The mixture was stirred at 20° C. for another 2 hrs. The mixture was quenched with NH4Cl and adjusted to pH>7 with aq. NaHCO3. The mixture was extracted three times with DCM. The combined organic layers

were dried over Na2SO4 and concentrate to give A9 (600 mg, crude) as a yellow solid. (TLC: N/A)

LCMS; Mass Calcd.: 612.3; MS Found: 612.4 [MS].

### Step 7) Synthesis of Compound D ((R)-18-(3,4-bis((4-fluorobenzyl)oxy)phenoxy)-1-(4-(4-(2,4-dibromo-1H-imidazol-1-yl)thiazol-2-yl)piperazin-1-yl)-3,6,9,12-tetraoxa-15-azaoctadecan-17-ol)

A mixture of A9 (600 mg, 0.98 mmol, 1.0 eq), B3 ((R)-2-((3,4-bis((4-

fluorobenzyl)oxy)phenoxy)methyl)oxirane, 390 mg, 0.98 mmol, 1.0 eq) and DIEA (253 mg, 1.96 mmol, 2.0 eq) in MeOH (5 mL) and MeCN (5 mL) was stirred at 60° C. for 16 hrs. The mixture was concentrated. The crude product was purified by prep-HPLC and lyophilized to give Compound D ((R)-18-(3,4-bis((4-fluorobenzyl)oxy)phenoxy)-1-(4-(4-(2,4-dibromo-1Himidazol-1-yl)thiazol-2-yl)piperazin-1-yl)-3,6,9,12-tetraoxa-15-azaoctadecan-17-ol, 58 mg, yield 5.6% for 3 steps from A7) as a yellow solid.

1H-NMR (CDCl3, 400 MHz): δ 7.36-7.43 (m, 5H), 7.02-7.09 (m, 4H), 6.85 (d, J=8.8 Hz, 1H), 6.65 (s, 1H), 6.60 (d, J=2.8 Hz, 1H), 6.40 (dd, J=8.8, 2.8 Hz, 1H), 5.08 (s, 2H), 5.02 (s, 2H), 4.42-4.44 (m, 1H), 4.00-4.03 (m, 1H), 3.86-3.94 (m, 3H), 3.64-3.74 (m, 14H), 3.52 (t, J=4.8 Hz, 4H), 3.26-3.30 (m, 2H), 3.07-3.19 (m, 2H), 2.67-2.68 (m, 6H).

LCMS; Mass Calcd.: 1010; MS Found: 1010.5 [MS+1].

### Example 5. Preparation of Compound E ((R)-18-(3,4-bis((4-fluorobenzyl)oxy)phenoxy)-1-(4-(4-(2,4-dibromo-1H-imidazol-1-yl)thiazol-2-yl)piperazin-1-yl)-17-hydroxy-3,6,9,12-tetraoxa-15-azaoctadecan-1-one)

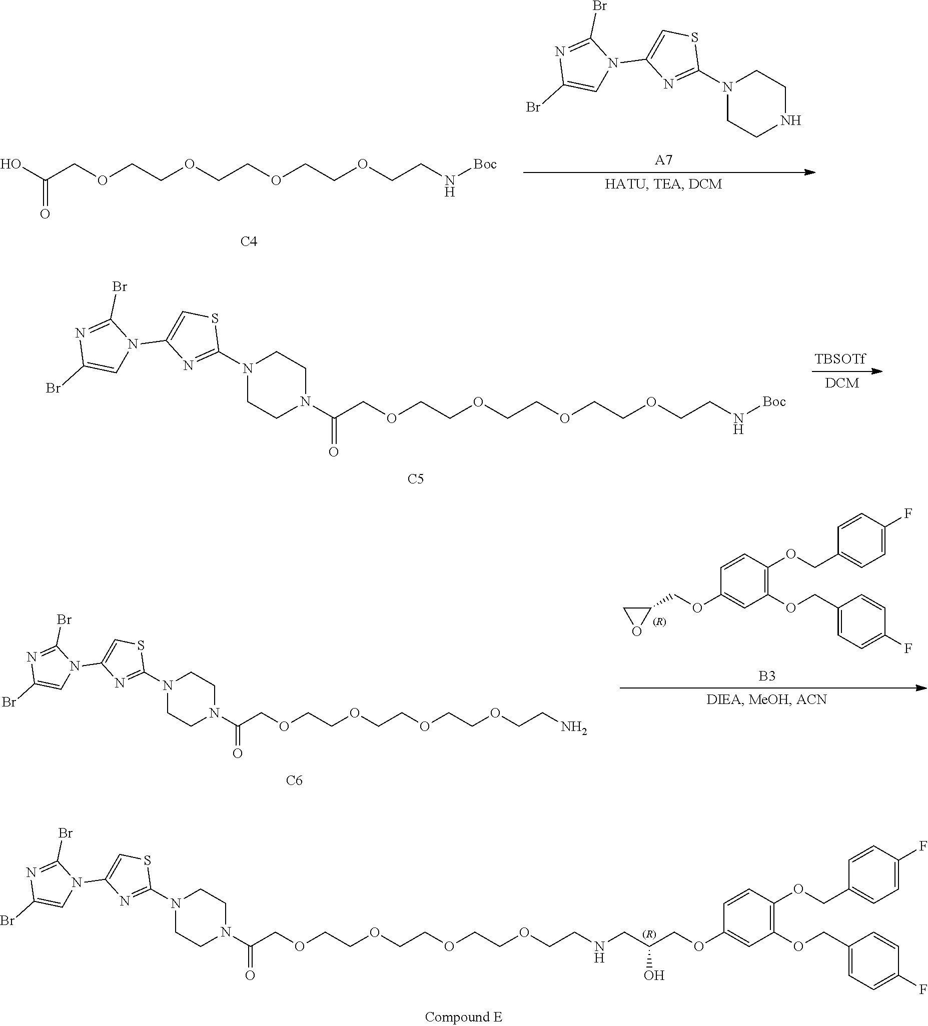

### Step 1) Synthesis of C5

To a solution of C4 (2,2-dimethyl-4-oxo-3,8,11,14,17-pentaoxa-5-azanonadecan-19-oic acid, 400 mg, 1.14 mmol, 1.0 eq), 4-(2,4-dibromo-1H-imidazol-1-yl)-2-(piperazin-1-yl)thiazole (A7, 448 mg, 1.14 mmol, 1.0 eq), HATU (563 mg, 1.48 mmol, 1.3 eq) and TEA (173 mg, 1.71 mmol, 1.5 eq) in DCM (10 mL) was stirred at 20° C. for 16 hrs. The mixture was poured into water and extracted with EA twice. The combined organic layers were washed with brine, dried over Na2SO4 and concentrated to obtain C5 (520 mg, crude) as yellow oil.

1H-NMR (CDCl3, 400 MHz): δ 7.40 (s, 1H), 6.73 (s, 1H), 4.27 (s, 2H), 3.60-3.78 (m, 14H), 3.50-3.55 (m, 8H), 3.31-3.33 (m, 2H), 1.45 (s, 9H).

### Step 2) Synthesis of C6

To a mixture of C5 (520 mg, 0.72 mmol, 1.0 eq), 2,6-dimethylpyridine (307 mg, 2.86 mmol, 4.0 eq) in DCM (10 mL) was added TBSOTf (552 mg, 2.14 mmol, 3.0 eq) at 20° C. The mixture was stirred at 20° C. for 2 hrs. Then another batch of TBSOTf (189 mg, 0.716 mmol, 1.0 eq) was added. The mixture was stirred at 20° C. for another 0.5 hr. Repeat this operation twice. The mixture was quenched with NH₄Cl and adjusted to pH=7˜8 with aq. NaHCO₃.

The mixture was extracted three times with DCM. The combined organic layers were washed with brine, dried over Na₂SO₄ and concentrate to give C6 (520 mg, crude) as a yellow solid. (TLC: DCM/MeOH=10/1, Rf=0.4)

LCMS; Mass Calcd.: 626.3; MS Found: 626.5 [MS].

### Step 3) Synthesis of Compound E ((R)-18-(3,4-bis((4-fluorobenzyl)oxy)phenoxy)-1-(4-(4-(2,4-dibromo-1H-imidazol-1-yl)thiazol-2-yl)piperazin-1-yl)-17-hydroxy-3,6,9,12-tetraoxa-15-azaoctadecan-1-one)

A mixture of C6 (350 mg, 0.56 mmol, 1.0 eq), (R)-2-((3,4-bis(4-fluorobenzyloxy)phenoxymethyl)oxirane (B3, 223 mg, 0.56 mmol, 1.0 eq) and DIEA (144 mg, 1.12 mmol, 2.0 eq) in MeOH (5 mL) and ACN (5 mL) was stirred at 60° C. for 16 hrs. The mixture was concentrated. The crude product was purified by prep-HPLC and lyophilized to give Compound E ((R)-18-(3,4-bis((4-fluorobenzyl)oxy)phenoxy)-1-(4-(4-(2,4-dibromo-1Himidazol-1-yl)thiazol-2-yl)piperazin-1-yl)-17-hydroxy-3,6,9,12-tetraoxa-15-azaoctadecan-1-one, 58 mg, yield 9.7%) as a white solid. (TLC: DCM/MeOH=10/1, Rf=0.5)

1H-NMR (CDCl3, 400 MHz): δ 8.53 (s, 1H), 7.36-7.44 (m, 5H), 7.02-7.09 (m, 4H), 6.85 (d, J=8.8 Hz, 1H), 6.70 (s, 1H), 6.61 (d, J=2.8 Hz, 1H), 6.42 (dd, J=8.8, 2.8 Hz, 1H), 5.08 (s, 2H), 5.02 (s, 2H), 4.32-4.39 (m, 3H), 4.03-4.06 (m, 1H), 3.84-3.93 (m, 3H), 3.64-3.75 (m, 14H), 3.59 (s, 4H), 3.50 (s, 2H), 3.12-3.27 (m, 4H).

LCMS; Mass Calcd.: 1024.1; MS Found: 1024.5 [MS].

### Example 6. Preparation of Compound F ((R)-4-(3-(4-cyano-3-(trifluoromethyl)phenyl)-5,5-dimethyl-4-oxo-2-thioxoimidazolidin-1-yl)-2-fluoro-N-(18-(3-((4-fluorobenzyl)oxy)phenoxy)-17-hydroxy-3,6,9,12-tetraoxa-15-azaoctadecyl)benzamide)

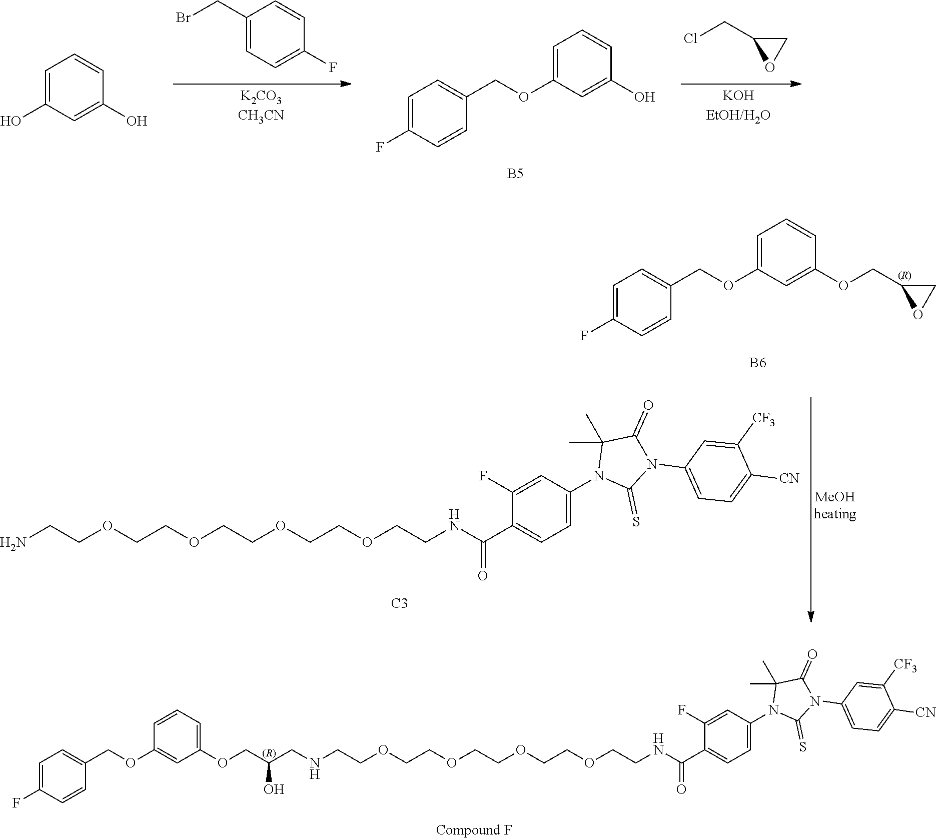

### Step 1) Synthesis of B5

To a solution of resorcinol (3 g, 27.24 mmol) and potassium carbonate (4.5 g, 32.6 mmol) in acetonitrile (16 mL), 1-(bromomethyl)-4-fluorobenzene (6.18 g, 32.6 mmol) was added then the mixture was heated to reflux and stirred for 14 hrs. After cooling to room temperature, water was added and the mixture was extracted with ethylacetate (EA). The organic phase was dried over Na2SO4 and concentrated. The residue was purified by column (Hexane:EA=10:1˜5:1) to afford B5 (3.25 g, 54.2% yield) as a yellow solid.

LCMS; Mass Calcd.: 218.23; MS Found: 219.0 [MS+1].

### Step 2) Synthesis of B6

To a solution of B5 (2 g, 9.16 mmol) in ethanol (EtOH, 20 ml), (R)-2-(chloromethyl)oxirane (2.09 g, 22.91 mmol) was added and then potassium hydroxide (KOH, 0.61 g, 10.99 mmol) in water (2 ML was added to the reaction mixture. The reaction mixture was stirred at room temperature for overnight. After quenched by water and then extracted with EA. The organic phase was washed with brine, and then dried over Na₂SO₄. The residue was concentrated in vacuo, purified by flash column chromatography (Hexane:DCM=8:1) to afford B6 (2.2 g, 88% yield) as a yellow oil.

LCMS; Mass Calcd.: 274.29; MS Found: 275.1 [MS+1].

### Step 3) Synthesis of Compound F (R)-4-(3-(4-cyano-3-(trifluoromethyl)phenyl)-5,5-dimethyl-4-oxo-2-thioxoimidazolidin-1-yl)-2-fluoro-(18-(3-((4-fluorobenzyl)oxy)phenoxy)-17-hydroxy-3,6,9,12-tetraoxa-15-azaoctadecyl)benzamide

A solution of B6 (66 mg, 0.24 mmol) and C3 (N-(14-amino-3,6,9,12-tetraoxatetradecyl)-4-(3-(4-cyano-3-(trifluromethyl)phenyl)-5,5-dimethyl-4-oxo-2-thioxoimidazolidin-1-yl)-2-fluorobenzamide, 170 mg, 0.25 mmol) in MeOH (8 ml) was stirred at 60° C. for 14 hrs. After the reaction was completed, the resulting mixture was cooled and evaporated to remove MeOH. The residue was purified by flash column chromatography (NH-silica; dichloromethane/MeOH=20/1) to give Compound F ((R)-4-(3-(4-cyano-3-(trifluoromethyl)phenyl)-5,5-dimethyl-4-oxo-2-thioxoimidazolidin-1-yl)-2-fluoro-N-(18-(3-((4-fluorobenzyl)oxy)phenoxy)-17-hydroxy-3,6,9,12-tetraoxa-15-azaoctadecyl)benzamide, 0.028 g, yield: 12.4%) as a light brown solid.

1H-NMR (DMSO-d6, 600 MHz) δ (ppm): δ 8.53 (s, 1H), 8.41 (d, 1H), 8.29 (s, 1H), 8.08 (d, 1H), 7.77 (t, 1H), 7.48-7.35 (m, 4H), 7.23-7.18 (m, 3H), 6.61-6.53 (m, 3H), 5.57 (br s, 1H), 5.06 (s, 2H), 4.04-4.01 (m, 1H), 3.91 (m, 2H), 3.68 (m, 2H), 3.53-3.51 (m, 11H), 3.43-3.3.34 (m, 6H) 2.97 (m, 2H), 2.85 (m, 1H), 1.54 (s, 6H).

LCMS; Mass Calcd.: 943.98, MS Found: 944.5. [MS+1].

### Example 7. Preparation of Compound G ((R)-4-(3-(4-cyano-3-(trifluoromethyl)phenyl)-5,5-dimethyl-4-oxo-2-thioxoimidazolidin-1-yl)-2-fluoro-N-(6-((5-((3-(3-((4-fluorobenzyl)oxy)phenoxy)-2-hydroxypropyl)amino)pentyl)oxy)hexyl)benzamide)

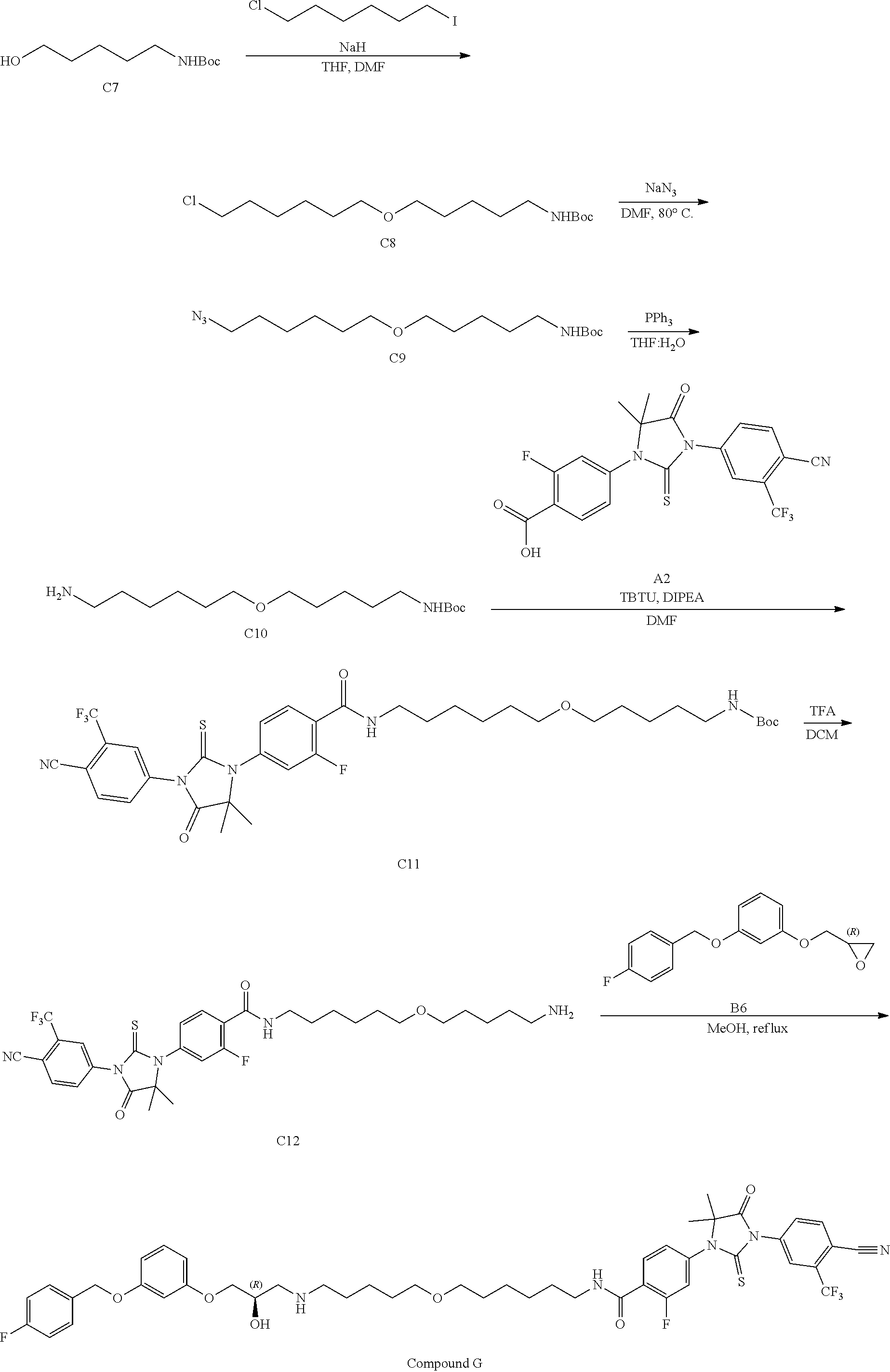

### Step 1) Synthesis of C8

To a solution of tert-butyl (5-hydroxypentyl)carbamate (C7, 0.83 g, 4.0 mmol) in anhydrous THF:anhydrous DMF=3:1 (16 mL) was added NaH (60% dispersion in Paraffin Liquid) (0.23 g, 5.8 mmol) at 0° C. After 30 mins. The reaction mixture was added 1-chloro-6-iodohexane (0.94 mL, 6.0 mmol) at 0° C. The mixture was stirred at 0° C. to room temperature for 15 hrs. After the reaction was completed, the mixture was quenched by NH4Cl solution (20 mL) at 0° C. The resulting mixture was extracted with ethyl acetate, washed by brine and dried over anhydrous MgSO4. The residue was concentrated in vacuo, purified by flash column chromatography (Hex/EA=2/1) to give C8 (0.49 g, yield: 37%) as a yellow oil.

ESI-MS Calcd m/z for C16H32CINO3 [M+H]+322.89 Found 322.

### Step 2) Synthesis of C9

To a solution of C8 (0.49 g, 1.5 mmol) in DMF (8 mL) was added NaN3 (0.20 g, 3.0 mmol). The mixture was stirred at 80° C. for 8 hrs. After the reaction was completed, the mixture was concentrated under reduced pressure. The residue was extracted with ethyl acetate, washed by brine and dried over anhydrous MgSO4. The residue was concentrated in vacuo to give C9 (0.5 g, theoretical yield: 100%) as a yellow oil.

ESI-MS Calcd m/z for C16H32N4O3 [M+H]+329.46 Found 329.

### Step 3) Synthesis of C10

To a solution of C9 (0.5 g, 1.5 mmol) in THF:H₄0=5:1 (18 mL) was added

triphenylphosphine (PPh3, 0.599 g, 2.3 mmol). The mixture was stirred at room temperature for 24 hrs. After the reaction was completed, the resulting mixture was evaporated to remove THF. The residue was dissolved in 2N HCl solution until pH 2.0. The aqueous solution was extracted by ethyl acetate (30 mL×3) then added 10% NaOH solution until pH 8.0. The aqueous solution was extracted by ethyl acetate (30 mL×3). Organic layer was washed with brine, dried over anhydrous MgSO4 and concentrated in vacuo to give C10 (0.262 g, yield: 57%) as an ivory solid.

ESI-MS Calcd m/z for C16H34N2O3 [M+H]+303.46 Found 303.

### Step 4) Synthesis of C11

To a solution of C10 (0.262 g, 0.86 mmol) in DMF (8 mL) was added TBTU (0.278 g, 0.86 mmol), N,N-diisopropylethylamine (DIPEA, 0.15 mL, 0.86 mmol) and 4-(3-(4-cyano-3-(trifluoromethyl)phenyl)-5,5-dimethyl-4-oxo-2-thioxoimidazolidin-1-yl)-2-fluorobenzoic acid (A2, 0.30 g, 0.66 mmol). The mixture was stirred at room temperature for 15 hrs. After the reaction was completed, the resulting mixture was evaporated to remove DMF. The residue was purified by flash column chromatography (DCM/MeOH=20/1) to give C11 (0.262 g, yield: 53%) as a white solid.

ESI-MS Calcd m/z for C36H45F4N5O5S [M+H]+ 736.84 Found 736.

### Step 5) Synthesis of C12

To a solution of C11 (0.262 g, 0.35 mmol) in DCM (10 mL) was added trifluoroacetic acid (TFA, 0.49 mL, 3.5 mmol). The mixture was stirred at room temperature for 5 hrs. After the reaction was completed, the resulting mixture was evaporated to remove TFA and then added ethyl ether. The formed precipitate was collected by filtration. The filter cake washed with ether and dried to give C12 (0.226 g).

ESI-MS Calcd m/z for C31H37F4N5O3S [M+H]+ 636.72 Found 636.

### Step 6) Synthesis of Compound G ((R)-4-(3-(4-cyano-3-(trifluoromethyl)phenyl)-5,5-dimethyl-4-oxo-2-thioxoimidazolidin-1-yl)-2-fluoro-N-(6-((5-((3-(3-((4-fluorobenzyl)oxy)phenoxy)-2-hydroxypropyl)amino) pentyl)oxy)hexyl)benzamide)

To a solution of C12 (0.226 g, 0.40 mmol) in MeOH (6 mL) was added 2-((3-((4-fluorobenzyl)oxy)phenoxy)methyl)oxirane (B6, 0.060 g, 0.22 mmol). The mixture was stirred at reflux for 5 hrs. After the reaction was completed, the resulting mixture was cooled and evaporated to remove MeOH. The residue was purified by flash column chromatography (NH-silica; dichloromethane/MeOH=10/1) to give Compound G ((R)-4-(3-(4-cyano-3-(trifluoromethyl)phenyl)-5,5-dimethyl-4-oxo-2-thioxoimidazolidin-1-yl)-2-fluoro-N-(6-((5-((3-(3-((4-fluorobenzyl)oxy)phenoxy)-2-hydroxypropyl)amino) pentyl)oxy)hexyl)benzamide, 0.036 g, yield: 18%) as a yellow solid.

1H-NMR (DMSO-d6, 600 MHz) δ (ppm): δ 8.50-8.49 (m, 1H), 8.40 (d, J=8.1 Hz, 1H), 8.29 (d, J=1.8 Hz, 1H), 8.08 (dd, J=8.2 Hz, J=1.8 Hz, 1H), 7.74 (t, J=8.0 Hz, 1H), 7.49-7.47 (m, 2H), 7.41 (d, J=10.5 Hz, 1H), 7.32 (dd, J=8.1 Hz, J=1.8 Hz, 1H), 7.23-7.19 (m, 2H), 7.18-7.15 (m, 1H), 6.59-6.56 (m, 2H), 6.53-6.51 (m, 1H), 5.06 (s, 2H), 3.93-3.83 (m, 3H), 3.34-3.29 (m, 6H), 3.26-3.22 (m, 2H), 2.57-2.56 (m, 2H), 1.57 (s, 6H), 1.54-1.46 (m, 6H), 1.45-1.40 (m, 2H), 1.32-1.30 (m, 6H).

ESI-MS Calcd m/z for C47H52F5N5O6S [M+H]+ 911.01 Found 911.

### Example 8. Preparation of Compound H (8-(4-(3-(4-cyano-3-(trifluoromethyl)phenyl)-5,5-dimethyl-4-oxo-2-thioxoimidazolidin-1-yl)phenoxy)-N-(2-(2-(2-((3-((4-fluorobenzyl)oxy)benzyl)amino)ethoxy)ethoxy)ethyl)octanamide

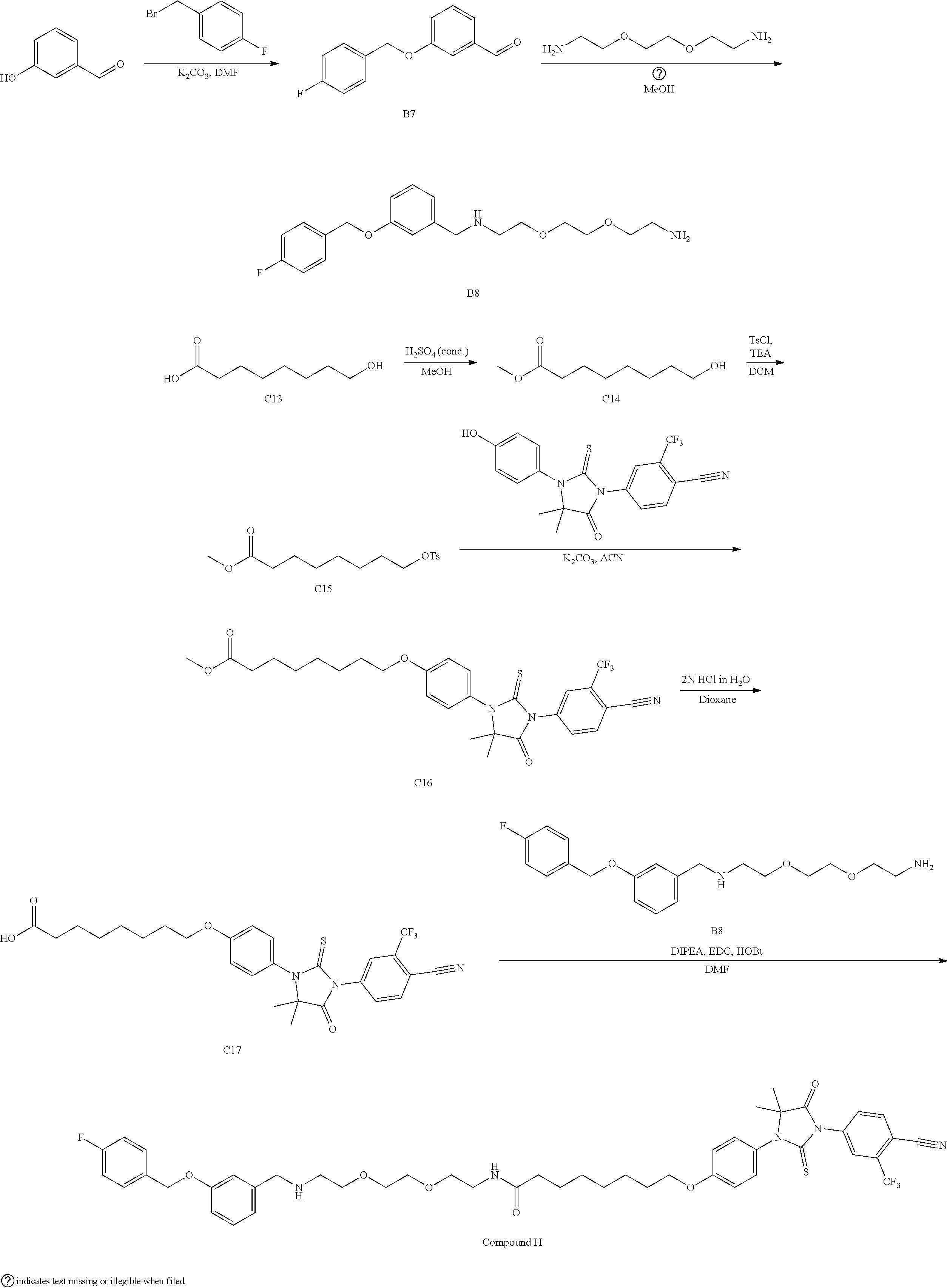

### Step 1) Synthesis of B7

To a solution of 3-hydroxybenzaldehyde (0.44 g, 3.60 mmol) in dimethylformamide (DMF) was added K2CO3 (0.5 g, 3.60 mmol) and 1-(bromomethyl)-4-fluorobenzene (0.50 ml, 4.32 mmol) at r.t. The mixture was stirred at 60° C. for 4 hours. The reaction mixture was cooled to r.t and extracted with ether and water. The organic layer was washed with brine, dried over MgSO₄, filtered and concentrated. The residue was purified by column chromatography on silica gel eluted with Ethyl acetate/Hexane (1:9) to afford B7 (3-((4-fluorobenzyl)oxy)benzaldehyde, 0.70 g, 84%).

1H-NMR (CDCl3, 400 MHz) δ (ppm) 9.98 (s, 1H), 7.48-7.40 (m, 5H), 7.25 (s, 1H), 7.11-7.02 (m, 2H), 5.08 (s, 2H)

ESI-MS Calcd m/z for C14H11FO2 [M+H]+ 231.10 Found 231.07.

### Step 2) Synthesis of B8

To a solution of B7 (25 g, 108.6 mmol) in MeOH (250 ml) was added 2,2′-(ethane-1,2-diylbis(oxy))bis(ethan-1-amine) (16.1 g, 108.6 mmol). The mixture was stirred for 6 hrs at 65° C. NaBH4 (4.14 g, 108.6 mmol) was added at r.t. The reaction mixture was stirred overnight at 50° C. The above solution was poured into water. The solution was extracted with ethyl acetate. The combined organic layers were washed with brine, dried over anhydrous Na2SO4 and concentrated. The crude was purified by column chromatography on silica gel (DCM/MeOH=12:1) to give B8 (2-(2-(2-aminoethoxy)ethoxy)-N-(3-((4-fluorobenzyl)oxy)benzyl)ethan-1-amine, 13 g, 33.0%) as yellow oil.

1H-NMR (DMSO+D2O, 400 MHz) δ (ppm) 7.49 (q, J=5.6 Hz, 2H), 7.21 (t, J=8.4 Hz, 3H), 6.97 (s, 1H), 6.89-6.75 (m, 2H), 5.05 (s, 21H), 3.65 (s, 2H), 3.48-3.44 (m, 6H), 3.34 (t, J=5.6 Hz, 2H), 2.59 (q, J=5.6 Hz, 4H).

ESI-MS Calcd m/z for C20H27FN2O3 [M+H]+ 362.20 Found 363.00.

### Step 3) Synthesis of C14

Compound C13 (194.4 mg, 1.21 mmol) was dissolved in methanol (24 ml) and conc. sulfuric acid (few drops) was added dropwise. Then the mixture was refluxed for 4 hrs. The mixture was evaporated and diluted with ethyl acetate. The crude was extracted with water and ethyl acetate, washed with sat, sodium bicarbonate and dried by anhydrous sodium sulfate. Then the mixture was filtered and concentrated to afford C14 (methyl 8-hydroxyoctanoate, 177.8 mg, 85.0%) as a yellow oil.

ESI-MS Calcd m/z for C9H18O3 [M+H]+ 175.13 Found 175.10.

### Step 4) Synthesis of C15

To a solution of C14 (76.6 mg, 0.44 mmol) in dichloromethane (1 ml) was added trimethylamine (TEA, 0.09 ml, 0.66 mmol) at 0° C. Then tosyl chloride was added at 0° C., followed by stirring of the mixture at r.t overnight. The mixture was diluted with dichloromethane and extracted with water and dichloromethane. The organic layer was dried anhydrous magnesium sulfate and concentrated. The crude was purified by column chromatography (Hex:EA=7:3) to afford C15 (methyl 8-(tosyloxy)octanoate, 105.9 mg, 73.0%) as a colorless oil.

ESI-MS Calcd m/z for C16H24O5S [M+H]+ 329.13 Found 329.20.

### Step 5) Synthesis of C16

To a solution of C15 (60.0 mg, 0.18 mmol) in acetonitrile (4.5 ml) was added 4-(3-(4-hydroxyphenyl)-4,4-dimethyl-5-oxo-2-thioxoimidazolidin-1-yl)-2-

(trifluoromethyl)benzonitrile (cas no. 915086-32-7, 63.0 mg, 0.16 mmol) and potassium carbonate (74.6 mg, 0.54 mmol). The mixture was refluxed overnight at 80° C. The reaction mixture was extracted with ethyl acetate and water and washed with brine. The organic layer was dried over sodium sulfate, filtered and concentrated. The crude was purified by column chromatography (Hex:EA=7:3) to afford C16 (methyl 8-(4-(3-(4-cyano-3-(trifluoromethyl)phenyl-5,5-dimethyl-4-oxo-2-thioxoimidazolidin-1-yl)phenoxy)octanoate, 205 mg, ˜99.0%) as a yellow oil.

ESI-MS Calcd m/z for C28H30F3N3O4S [M+H]+ 561.19 Found 561.00.

### Step 6) Synthesis of C17

To a solution of C16 (205 mg, 0.37 mmol) in 1,4-dioxane (4.5 ml) was added 2N HCl (aq., 1 ml) dropwise. The reaction mixture was stirred at 80° C. for 2 hrs. The resulting mixture was concentrated to afford C17 (8-(4-(3-(4-cyano-3-(trifluoromethyl)phenyl)-5,5-dimethyl-4-oxo-2-thioxoimidazolidin-1-yl)phenoxy)octanoic acid, 191.8 mg, 95.0%).

ESI-MS Calcd m/z for C27H28F3N3O4S [M+H]+ 548.18 Found 548.20.

### Step 7) Synthesis of Compound H (8-(4-(3-(4-cyano-3-(trifluoromethyl)phenyl-5,5-dimethyl-4-oxo-2-thioxoimidazolidin-1-yl)phenoxy)-N-(2-(2-(2-((3-((4-

fluorobenzyl)oxy)benzyl)amino)ethoxy)ethoxy)ethyl)octanamide)

To a solution of C17 (107.4 mg, 0.20 mmol) in dimethylformamide (DMF, 4 ml) was added B8 (90.6 mg, 0.25 mmol), N,N-diisopropylethylamine (DIPEA, 0.16 ml), 1-ethyl-3-(3-dimethylaminopropyl)carbodiimide (EDCI, 76.7 mg, 0.40 mmol) and Hydroxybenzotriazole (HOBT, 61.3 mg, 0.40 mmol). The reaction mixture was stirred at r.t for 5 hrs. After the reaction, water was added to the reaction and the crude was extracted with ethyl acetate and water. The organic layers were washed with brine, dried over anhydrous magnesium sulfate, filtered and concentrated. The residue was purified by column chromatography (DCM:MeOH=95:5) to afford Compound H (8-(4-(3-(4-cyano-3-(trifluoromethyl)phenyl)-5,5-dimethyl-4-oxo-2-thioxoimidazolidin-1-yl)phenoxy)-N-(2-(2-(2-((3-((4-fluorobenzyl)oxy)benzyl)amino)ethoxy)ethoxy)ethyl)octanamide, 56.7 mg, 40.0%) as a yellow solid.

1H-NMR (DMSO-d6, 600 MHz) δ (ppm) 8.38 (d, J=8.4 Hz, 1H), 8.28 (d, J=1.8 Hz, 1H), 8.06 (dd, J=2.4, 8.4 Hz, 1H), 7.81 (t, J=6 Hz, 1H), 7.47-7.50 (m, 2H), 7.26-7.27 (m, 2H), 7.19-7.23 (m, 3H), 7.06-7.07 (m, 2H), 7.00 (s, 1H), 6.90 (d, J=7.2 Hz, 1H), 6.86 (dd, J=1.2, 8.4 Hz, 1H), 5.06 (s, 2H), 3.99 (t, J=1.2 Hz, 2H), 3.72 (br s, 2H), 3.46-3.49 (m, 6H), 3.38 (t, J=6 Hz, 2H), 3.16 (q, J=5.8 Hz, 2H), 2.65 (br s, 2H), 2.04 (t, J=7.8 Hz, 2H), 1.69-1.74 (m, 2H), 1.46-1.49 (m, 7H), 1.38-1.43 (m, 2H), 1.29-1.34 (m, 2H), 1.21-1.27 (m, 2H).

ESI-MS Calcd m/z for C47H53F4N5O6S [M+H]+ 892.37 Found 893.00.

### Example 9. Preparation of Compound I (4-(3-(4-((1-(3-((4-fluorobenzyl)oxy)phenyl)-5,8,11,14,17-pentaoxa-2-azanonadecan-19-yl)oxy)phenyl)-4,4-dimethyl-5-oxo-2-thioxoimidazolidin-1-yl)-2-(trifluoromethyl)benzonitrile

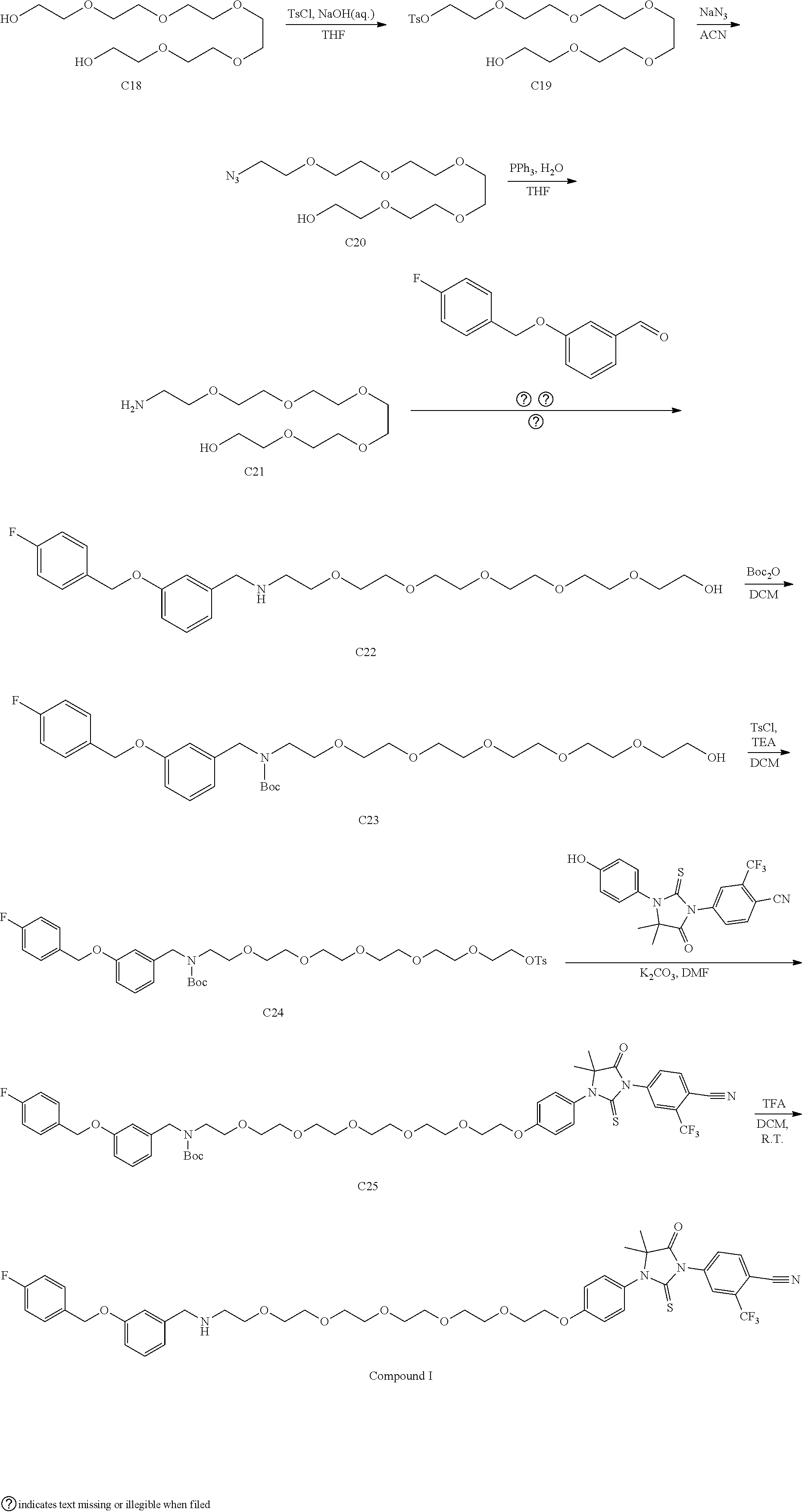

### Step 1) Synthesis of C19

To a solution of C18 (3,9,12,15-pentaoxaheptadecane-1,17-diol, 3.305 g, 10.8 mmol) in tetrahydrofuran (THF, 12 ml) was added aq. NaOH (2 ml) at 0° C. After 30 min, a solution of tosyl chloride in THF (10 ml) was added to the reaction mixture at 0° C. dropwise over 1 hr. The reaction mixture was warmed slowly to r.t and stirred overnight. The mixture was concentrated, diluted with dichloromethane and extracted with dichloromethane and water. The organic layers were washed with brine, dried over anhydrous magnesium sulfate, filtered and concentrated. The residue was purified by column chromatography (DCM:MeOH=95:5) to afford C19 (17-hydroxy-3,6,9,12,15-pentaoxaheptadecyl 4-methylbenzenesulfonate, 1.62 g,

35.0%) as a colorless oil.

ESI-MS Calcd m/z for C19H32O9S [M+H]+ 437.18 Found 437.10.

### Step 2) Synthesis of C20

Compound C19 (1.62 g, 3.71 mmol) was dissolved in acetonitrile (ACN, 37 ml) and to the mixture was added sodium azide (481.9 mg, 7.41 mmol). The reaction mixture was refluxed at 80° C. for 6 hrs. The solvent was concentrated and ether was added. The solids were filtered and washed. Ether was concentrated to afford C20 (17-azido-3,6,9,12,15-pentaoxaheptadecan-1-ol, 721.1 mg, 63.2%) as a yellow oil.

ESI-MS Calcd m/z for C12H25N3O6 [M+H]+ 308.17 Found 307.90.

### Step 3) Synthesis of C21

To a solution of C20 (721.1 mg, 2.35 mmol) in tetrahydrofuran (THF, 8 ml) was added PPh3 (1.23 g, 4.69 mmol) at r.t. After 10 min, water was added and stirred at r.t for overnight. The solvent was concentrated. Then the residue was purified by amine column chromatography (DCM:MeOH=95:5˜0:100) to afford C21 (17-amino-3,6,9,12,15-pentaoxaheptadecan-1-ol, 552.8 mg, 83.0%) as a yellow oil.

ESI-MS Calcd m/z for C12H27NO6 [M+H]+ 282.18 Found 282.30.

### Step 4) Synthesis of C22

To a solution of C21 (300.0 mg, 1.07 mmol) in ethanol (EtOH, 10 ml) was added B7 (3-((4-fluorobenzyl)oxy)benzaldehyde, 163.67 mg, 0.71 mmol) in EtOH (10 ml). The reaction mixture was refluxed at 65° C. for overnight. After the reaction mixture was cooled to r.t, sodium borohydride (53.7 mg, 1.42 mmol) was added and stirred for 3 hrs. The solvent was concentrated and extracted with ethyl acetate and water. The organic layers were dried over anhydrous magnesium sulfate, filter and concentrated. The residue was purified by amine

column chromatography (DCM:MeOH=95:5˜0:100) to afford C22 (1-(3-((4-fluorobenzyl)oxy)phenyl)-5,8,11,14,17-pentaoxa-2-azanonadecan-19-ol, 209.6 mg, 60.0%).

ESI-MS Calcd 495.26, Found 496.10.

### Step 5) Synthesis of C23

A solution of di-tert-butyl dicarbonate (Boc2O, 91.7 mg, 0.42 mmol) in dichloromethane (1 ml) was added dropwise to a stirring solution of C22 (209.6 mg, 0.42 mmol) in dry dichloromethane (1 ml) at 0° C. The reaction mixture was stirred for 30 min at 0° C., and then at r.t for overnight. The mixture was diluted with dichloromethane, washed with sat, sodium bicarbonate and water. The organic layers were dried over anhydrous sodium sulfate, filtered and concentrated. The residue was purified by column chromatography (DCM:MeOH=95:5) to afford C23 (tert-butyl (3-((4-fluorobenzyl)oxy)benzyl)(17-hydroxy-3,6,9,12,15-pentaoxaheptadecyl)carbamate, 164.9 mg, 66.0%).

ESI-MS Calcd 595.32 Found 596.10.

### Step 6) Synthesis of C24

To a solution of C23 (164.9 mg, 0.28 mmol) in dichloromethane (1 ml) was added trimethylamine (TEA, 0.06 ml) and cooled to 0° C. Tosyl chloride (52.7 mg, 0.28 mmol) was added to the solution, followed by stirring of the mixture at r.t overnight. The reaction mixture was diluted with dichloromethane and extracted. The organic layers were dried by anhydrous magnesium sulfate, filtered and concentrated. The residue was purified by column chromatography (DCM:MeOH=95:5) to afford C24 (5-(3-((4-fluorobenzyl)oxy)benzyl)-2,2-dimethyl-4-oxo-3,8,11,14,17,20-hexaoxa-5-azadocosan-22-yl 4-methylbenzenesulfonate, 172.9 mg, 83.0%) as a yellow oil.

ESI-MS Calcd 749.32 Found 750.40.

### Step 7) Synthesis of C25

To a solution of C24 (172.9 mg, 0.23 mmol) in acetonitrile (ACN, 4.5 ml) was added 4-(3-(4-hydroxyphenyl)-4,4-dimethyl-5-oxo-2-thioxoimidazolidin-1-yl)-2-(trifluoromethyl)benzonitrile (cas no. 915086-32-7, 93.5 mg, 0.23 mmol), potassium carbonate (K2CO3, 95.4 mg, 0.69 mmol). The reaction mixture was refluxed at 80° C. for overnight. The mixture was extracted with ethyl acetate and water and washed with brine. The organic layers were dried over anhydrous sodium sulfate, filtered and concentrated. The residue was purified by amine column chromatography (DCM:MeOH=95:5) to afford C25 (tert-butyl (17-(4-(3-(4-cyano-3-(trifluoromethyl)phenyl)-5,5-dimethyl-4-oxo-2-thioxoimidazolidin-1-yl)phenoxy)-3,6,9,12,15-pentaoxaheptadecyl)(3-((4-fluorobenzyl)oxy)benzyl)carbamate, 317.6 mg, 50.0%) as a yellow powder.

ESI-MS Calcd 982.38 Found 983.20.

### Step 8) Synthesis of Compound I (4-(3-(4-((1-(3-((4-fluorobenzyl)oxy)phenyl)-5,8,11,14,17-pentaoxa-2-azanonadecan-19-yl)oxy)phenyl)-4,4-dimethyl-5-oxo-2-thioxoimidazolidin-1-yl)-2-(trifluoromethyl)benzonitrile)

To a solution of C25 (105.4 mg, 0.11 mmol) in dichloromethane (1 ml) was added trifluoroacetic acid (TFA, 0.08 ml) at r.t. The reaction mixture was stirred overnight. The solvent was concentrated and the residue was purified by column chromatography (DCM:MeOH=95:5) to afford Compound I (4-(3-(4-((1-(3-((4-fluorobenzyl)oxy)phenyl)-5,8,11,14,17-pentaoxa-2-azanonadecan-19-yl)oxy)phenyl)-4,4-dimethyl-5-oxo-2-thioxoimidazolidin-1-yl)-2-(trifluoromethyl)benzonitrile, 64.5 mg, 66.0%).

1H-NMR (DMSO-d6, 600 MHz) δ (ppm) 8.38 (d, J=8.0 Hz, 1H), 8.29 (s, 1H), 8.07 (d, J=8.1 Hz, 1H), 7.49 (s, 2H), 7.27 (d, J=6.9 Hz, 2H), 7.20 (t, J=8.1 Hz, 3H), 7.10 (d, J=7.5 Hz, 2H), 6.97 (s, 1H), 6.88 (d, J=7.2 Hz, 1H), 6.84 (d, J=7.8 Hz, 1H), 5.05 (s, 2H), 4.14 (s, 2H), 3.76 (s, 2H), 3.66 (s, 2H), 3.59 (s, 2H), 3.55-3.43 (m, 13H), 2.60 (s, 2H), 1.48 (s, 5H).

ESI-MS Calcd m/z for C45H50F4N4O8S [M+H]+ 883.33 Found 883.20.

Hereinafter, experimental examples to show the effects of the disclosed compounds are described. Unless otherwise described, the following experiments were conducted with conventional materials and methos well known in the an for measuring a similar function.

### Experimental Example 1. Increase of the Autophagy Flux in Androgen-Responsive and Unresponsive Cell by AR Inhibitors of the Present Invention

### 1. AUTOTAC is a Potent Autophagy Activator

Xtandi-AUTOTAC (Compounds A, B and C) efficiently increase autophagy at low concentration (0.01 μM).
Xtandi-AUTOTAC (Compound B) efficiently increases autophagy at early time point (1 h).

### 2. Substrate Aggregation Via p62

Xtandi-AUTOTAC promotes aggregation of specific substrate to inactivate it (C).

### 3. Long-Lasting Effect

Xtandi-AUTOTAC can keep autophagy active for up to 72 h (D).

### 4. Selectivity

Xtandi-AUTOTAC increase autophagy depend on p62 (E).

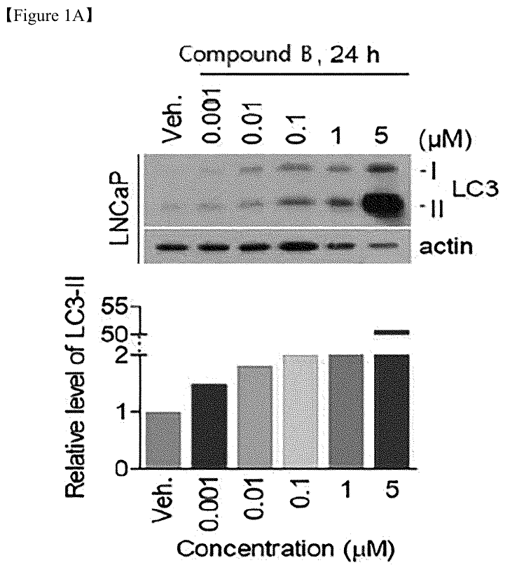

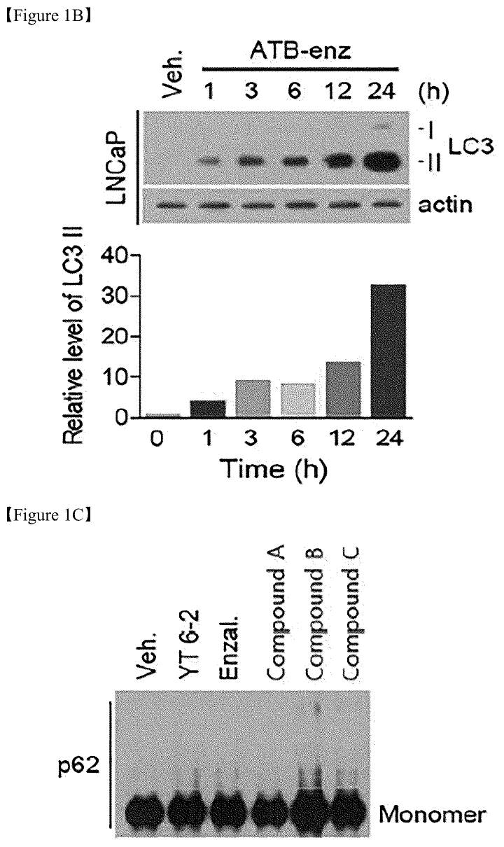

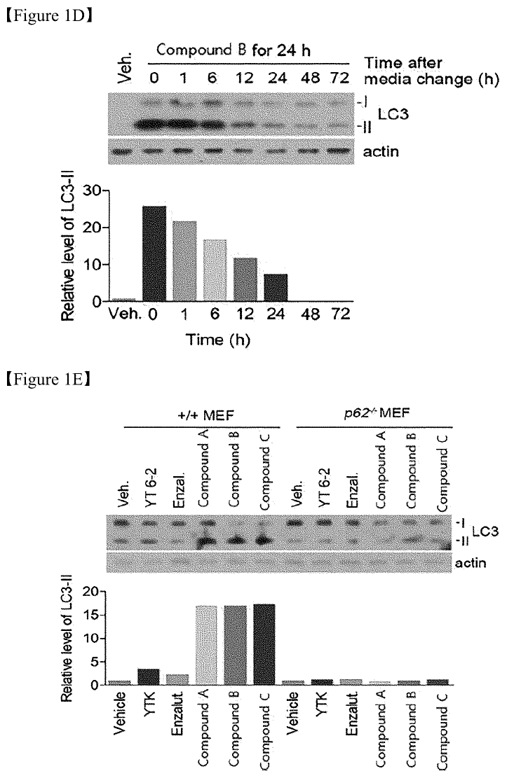

Please see the FIG. 1a to FIG. 1e.

### Experimental Example 2. Anticancer Efficacy Via Degradation of Androgen Receptor

Xtandi vs. AUTOTAC (Present Invention)

### 1. Degradation of AR

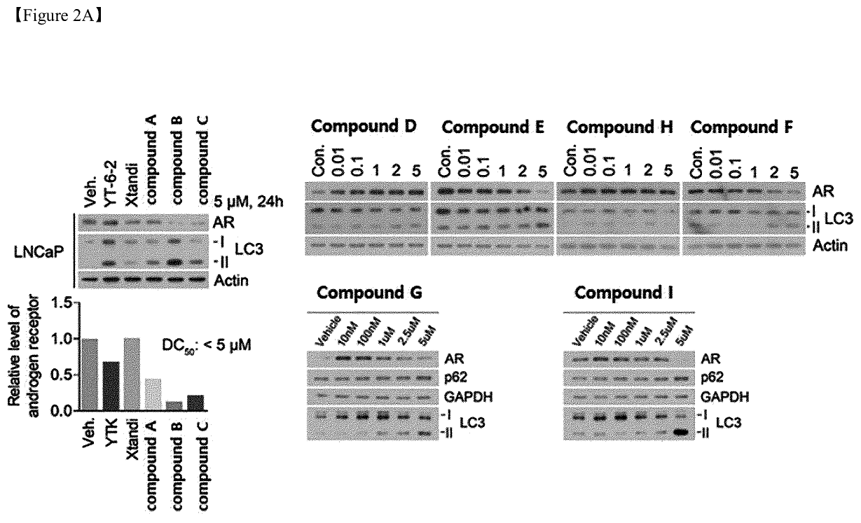

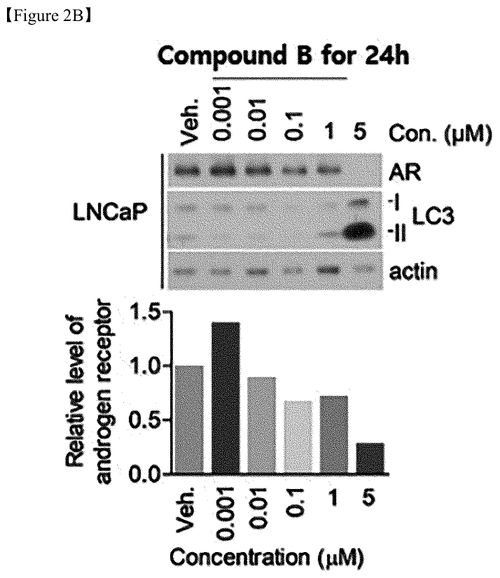

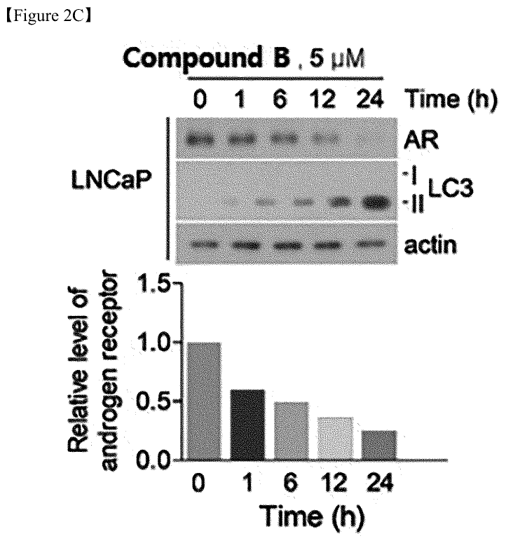

All Xtandi-AUTOTAC compounds (Compounds, A, B, C, D, E, F, G, H and I) efficiently degrade androgen receptor through autophagy (See FIG. 2a to FIG. 2c)

### 2. Inactivation of AR

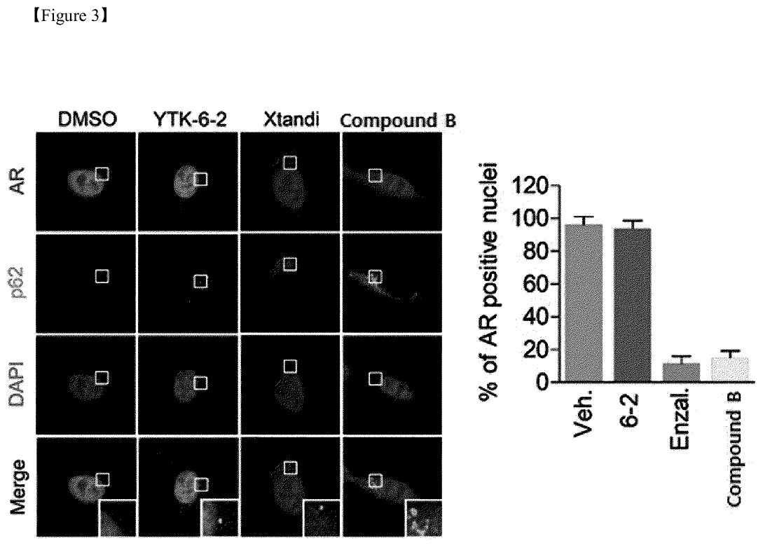

Compound B inhibits transcriptional activity of androgen receptor by forming aggregation with p62 in the cytosol. (See FIG. 3)

### 3. Down-Regulation of AR Signaling Pathway

Androgen receptor signaling is a critical pathway for prostate cancer cells
Compound B down-regulates expression of AR target genes

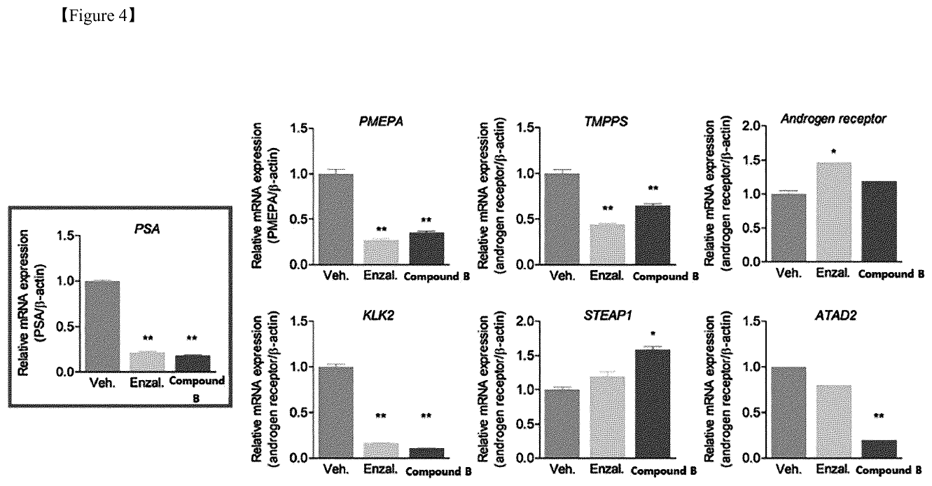

See FIG. 4.

### 4. Induce Prostate Cancer Cell Death

Treatment of Xtandi-AUTOTAC induces prostate cancer cell death.

See FIG. 5.

### Experimental Example 3. Compound B Inhibits Proliferation of Androgen-Responsive Prostate Cancer Cell

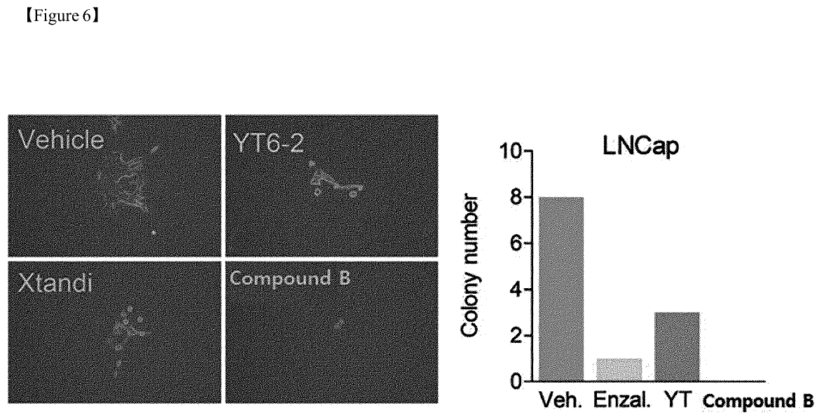

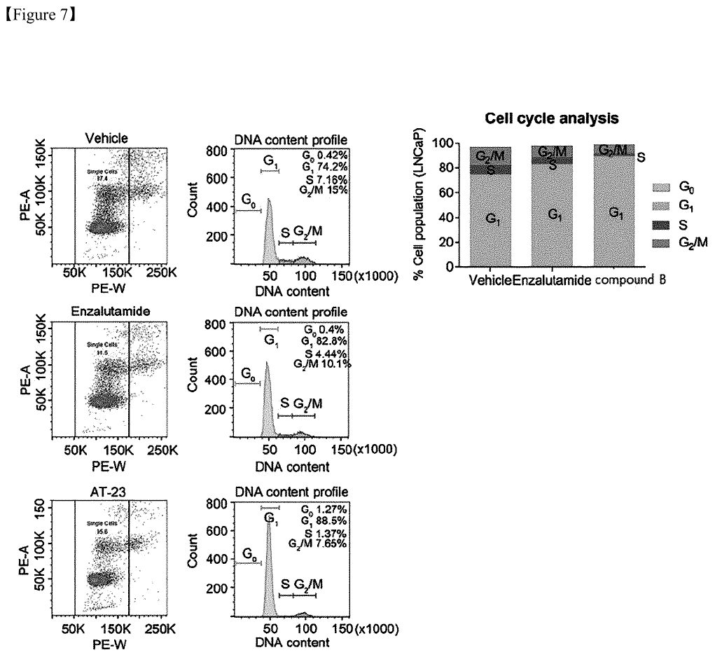

1. Compound B prevents colony formation of prostate cancer cell (See FIG. 6).
2. Compound B inhibits cell proliferation by blocking the cell cycle in G1 phase (See FIG. 7).

### Experimental Example 4. Xtandi-AUTOTAC Inhibits the Growth of LNCaP Xenograft Tumors in Mice Significantly

### 1. Inhibition of Tumor Growth

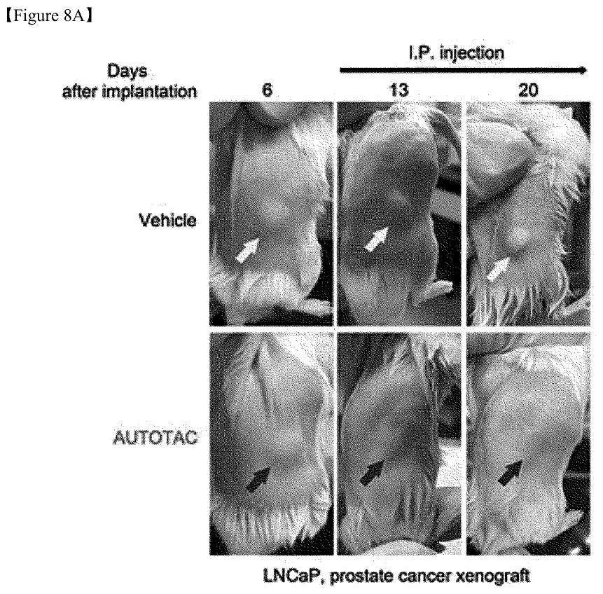

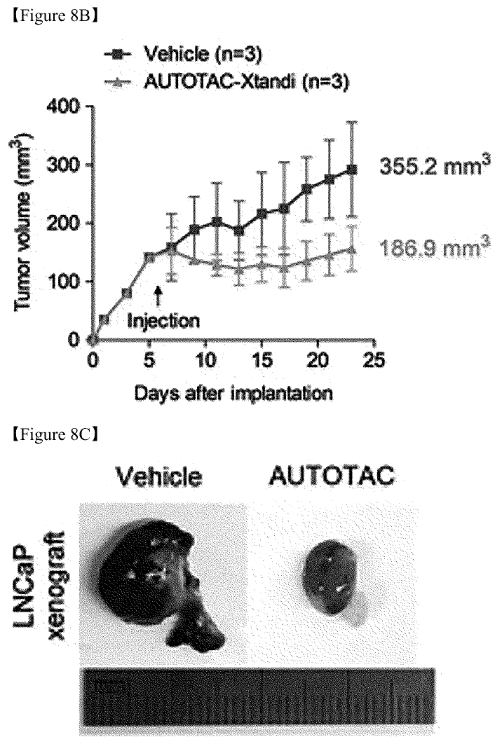

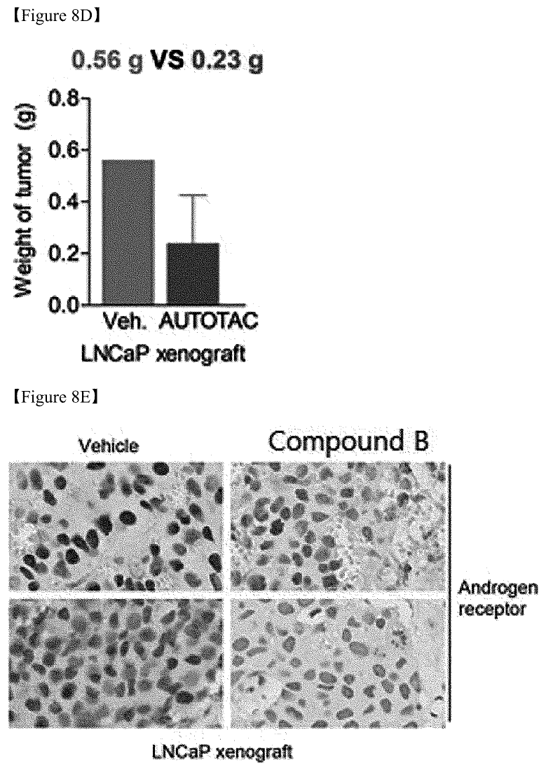

Compound B efficiently inhibits tumor growth in Xenograft mouse model (FIG. 8a to 8d).

### 2. Degradation of Androgen Receptor

Compound B degrades androgen receptor in tumor of mice (FIG. 8e).

### Experimental Example 5. Xtandi-AUTOTAC Degrades AR Splice Variant 7, Predictive Biomarker of Metastatic Castration-Resistant Prostate Cancer

The PROTAC has the limitation which it cannot degrade the AR-V7 and It is still needs to high unmet medical needs for AR-V7 targeted treatment of prostate cancer

Wt AR homodimer can be inhibited by Xtandi in the wt prostate cancer, however it cannot bind to AR-V7.

The PROTAC degrader can degrade the Wt AR-V7 heterodimer in principle. However the the data shows it is hard to degrade it because the internal diameter of proteasome (Arvinas Clinical Program Update ARV-471&ARV-110 on homepage, 14 Dec. 2020).

AUTOTAC has the advantage that induce the co-degradation by lysosome through the copolymerization-autophagic co-targeting by p62, unlike the PROTAC approach.

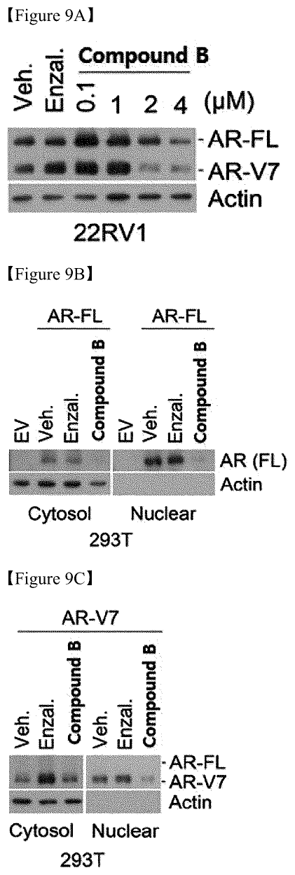

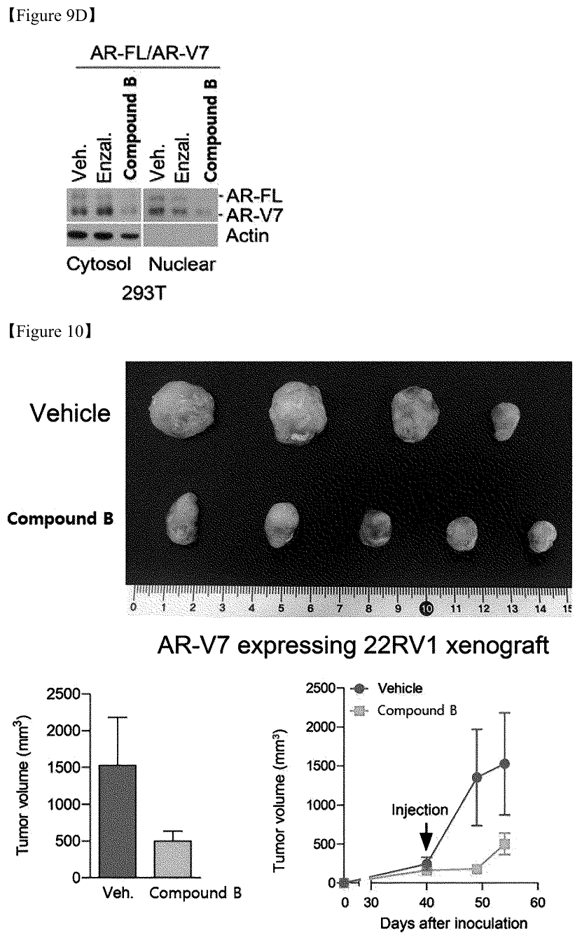

### Degradation of AR Variant (FIG. 9a to FIG. 9d)

AR splice variant 7 (AR-V7) mediates Xtandi(Enzalutamide) resistance.
Compound B degrades AR-V7 as well as wild type AR in enzalutamide resistant cell (FIG. 9a).
Compound B can degrade AR-V7 by sequestering with wild type AR.

### Experimental Example 6. Compound B Inhibits Tumor Growth in AR-V7-Expressing 22Rv1 Xenograft Model

22Rv1 cells (4×106 cells/animal) were injected subcutaneously into 10-week-old male SCID mice to generate xenograft. The mice bearing tumor were randomly divided into vehicle or AUTOTAC treatment groups (4 or 5 mice per group), Compound B was injected via intraperitoneally 5 times per week at a dose of 20 mg/kg body weight, whereas the vehicle group received an equal volume of PBS containing 5% DMSO and 10% solutol. (FIG. 10)

1. A compound of Formula (I), pharmaceutically acceptable salt, diastereomers, enantiomers, racemates, tautomers, prodrugs, hydrates, or solvates thereof: 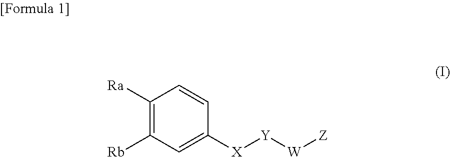
in formula (I),
wherein Ra and Rb are independently H or —O—CH2-R1,
R1 is C6-C9 aryl optionally substituted with one or more halos,
X is —O—CH2-CH(OH)—CH2-NH— or —CH2-NH—;
Y is —(CH2CH2-O)n1-, —(CH2)n2-O—, n1 and n2 are independently an integer of 1 to 6;
W is —(CH2)n3-NH—C(═O)—, —(CH2)n4-NH—C(═O)—(CH2)n5-O—, —CH2-CH2-O—, —CH2-C(═O)—, n3, n4 and n5 are independently an integer of 1 to 7; and
Z is

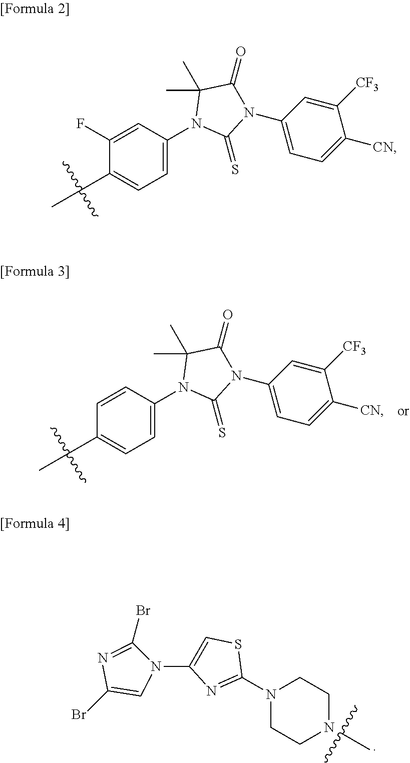

2. The compound, pharmaceutically acceptable salt, diastereomers, enantiomers, racemates, tautomers, prodrugs, hydrates, or solvates thereof according to claim 1, wherein Ra and Rb are independently H or benzyloxy optionally substituted with one or two halogens.

3. The compound, pharmaceutically acceptable salt, diastereomers, enantiomers, racemates, tautomers, prodrugs, hydrates, or solvates thereof according to claim 1, wherein Ra and Rb are H or 4-fluorobenzyloxy.

4. The compound, pharmaceutically acceptable salt, diastereomers, enantiomers, racemates, tautomers, prodrugs, hydrates, or solvates thereof according to claim 1, wherein Y is —(CH2CH2-O)n1-, —(CH2)n2-O—, n1 and n2 are independently an integer of 1 to 6.

5. The compound, pharmaceutically acceptable salt, diastereomers, enantiomers, racemates, tautomers, prodrugs, hydrates, or solvates thereof according to claim 1, wherein, W is —CH2CH2-NH—C(═O)—, —CH2CH2-O—, —CH2-C(═O)—, —(CH2)6-NH—C(═O)—, —(CH2)2-NHC(═O)—(CH2)7-O—.

6. The compound, pharmaceutically acceptable salt, diastereomers, enantiomers, racemates, tautomers, prodrugs, hydrates, or solvates thereof according to claim 1, wherein the compound is selected from the group consisting of the compounds below:
(R)—N-(2-(2-(2-((3-(3,4-bis((4-fluorobenzyl)oxy)phenoxy)-2-hydroxypropyl)amino)ethoxy)ethoxy)ethyl)-4-(3-(4-cyano-3-(trifluoromethyl)phenyl)-5,5-dimethyl-4-oxo-2-thioxoimidazolidin-1-yl)-2-fluorobenzamide;
(R)—N-(18-(3,4-bis((4-fluorobenzyl)oxy)phenoxy)-17-hydroxy-3,6,9,12-tetraoxa-15-azaoctadecyl)-4-(3-(4-cyano-3-(trifluoromethyl)phenyl)-5,5-dimethyl-4-oxo-2-thioxoimidazolidin-1-yl)-2-fluorobenzamide;
(R)—N-(24-(3,4-bis((4-fluorobenzyl)oxy)phenoxy)-23-hydroxy-3,6,9,12,15,18-hexaoxa-21-azatetracosyl)-4-(3-(4-cyano-3-(trifluoromethyl)phenyl)-5,5-dimethyl-4-oxo-2-thioxoimidazolidin-1-yl)-2-fluorobenzamide;
(R)-18-(3,4-bis((4-fluorobenzyl)oxy)phenoxy)-1-(4-(4-(2,4-dibromo-1H-imidazol-1-yl)thiazol-2-yl)piperazin-1-yl)-3,6,9,12-tetraoxa-15-azaoctadecan-17-ol;
(R)-18-(3,4-bis((4-fluorobenzyl)oxy)phenoxy)-1-(4-(4-(2,4-dibromo-1H-imidazol-1-yl)thiazol-2-yl)piperazin-1-yl)-17-hydroxy-3,6,9,12-tetraoxa-15-azaoctadecan-1-one;
(R)-4-(3-(4-cyano-3-(trifluoromethyl)phenyl)-5,5-dimethyl-4-oxo-2-thioxoimidazolidin-1-yl)-2-fluoro-N-(18-(3-((4-fluorobenzyl)oxy)phenoxy)-17-hydroxy-3,6,9,12-tetraoxa-15-azaoctadecyl)benzamide;
(R)-4-(3-(4-cyano-3-(trifluoromethyl)phenyl)-5,5-dimethyl-4-oxo-2-thioxoimidazolidin-1-yl)-2-fluoro-N-(6-((5-((3-(3-((4-fluorobenzyl)oxy)phenoxy)-2-hydroxypropyl)amino)pentyl)oxy)hexyl)benzamide;
8-(4-(3-(4-cyano-3-(trifluoromethyl)phenyl)-5,5-dimethyl-4-oxo-2-thioxoimidazolidin-1-yl)phenoxy)-N-(2-(2-(2-((3-((4-fluorobenzyl)oxy)benzyl)amino)ethoxy)ethoxy)ethyl)octanamide; and
4-(3-(4-((1-(3-((4-fluorobenzyl)oxy)phenyl)-5,8,11,14,17-pentaoxa-2-azanonadecan-19-yl)oxy)phenyl)-4,4-dimethyl-5-oxo-2-thioxoimidazolidin-1-yl)-2-(trifluoromethyl)benzonitrile.

7. A method of treating androgen receptor related disease in a subject in need thereof, comprising administering to said subject a therapeutically effective amount of the compound of claim 1, or pharmaceutically acceptable salt, diastereomers, enantiomers, racemates, tautomers, prodrugs, hydrates, or solvates thereof.

8. The method of claim 7, the androgen receptor related disease is prostate cancer.

9. The method of claim 7, the prostate cancer is castration resistant prostate cancer.

10. A method of inhibiting androgen receptor selectively, in biological sample or in a patient, comprising contacting the biological sample with or administering to a patient the compound of claim 1, pharmaceutically acceptable salt, diastereomers, enantiomers, racemates, tautomers, prodrugs, hydrates, or solvates thereof.

11. A pharmaceutical composition comprising the compound of claim 1, pharmaceutically acceptable salt, diastereomers, enantiomers, racemates, tautomers, prodrugs, hydrates, or solvates thereof.

12-14. (canceled)

15. A method of treating androgen receptor related disease in a subject in need thereof, comprising administering to said subject a therapeutically effective amount of the compound of claim 2, or pharmaceutically acceptable salt, diastereomers, enantiomers, racemates, tautomers, prodrugs, hydrates, or solvates thereof.

16. A method of treating androgen receptor related disease in a subject in need thereof, comprising administering to said subject a therapeutically effective amount of the compound of claim 3, or pharmaceutically acceptable salt, diastereomers, enantiomers, racemates, tautomers, prodrugs, hydrates, or solvates thereof.

17. A method of treating androgen receptor related disease in a subject in need thereof, comprising administering to said subject a therapeutically effective amount of the compound of claim 4, or pharmaceutically acceptable salt, diastereomers, enantiomers, racemates, tautomers, prodrugs, hydrates, or solvates thereof.

18. A method of treating androgen receptor related disease in a subject in need thereof, comprising administering to said subject a therapeutically effective amount of the compound of any of claim 5, or pharmaceutically acceptable salt, diastereomers, enantiomers, racemates, tautomers, prodrugs, hydrates, or solvates thereof.

19. A method of treating androgen receptor related disease in a subject in need thereof, comprising administering to said subject a therapeutically effective amount of the compound of claim 6, or pharmaceutically acceptable salt, diastereomers, enantiomers, racemates, tautomers, prodrugs, hydrates, or solvates thereof.

20. A method of inhibiting androgen receptor selectively, in biological sample or in a patient, comprising contacting the biological sample with or administering to the patient a compound of claim 2, pharmaceutically acceptable salt, diastereomers, enantiomers, racemates, tautomers, prodrugs, hydrates, or solvates thereof.

21. A method of inhibiting androgen receptor selectively, in biological sample or in a patient, comprising contacting the biological sample with or administering to the patient a compound of claim 3, pharmaceutically acceptable salt, diastereomers, enantiomers, racemates, tautomers, prodrugs, hydrates, or solvates thereof.

22. A method of inhibiting androgen receptor selectively, in biological sample or in a patient, comprising contacting the biological sample with or administering to the patient a compound of claim 4, pharmaceutically acceptable salt, diastereomers, enantiomers, racemates, tautomers, prodrugs, hydrates, or solvates thereof.

23. A method of inhibiting androgen receptor selectively, in biological sample or in a patient, comprising contacting the biological sample with or administering to the patient a compound of claim 5, pharmaceutically acceptable salt, diastereomers, enantiomers, racemates, tautomers, prodrugs, hydrates, or solvates thereof.
